SEQUENCE LISTING (75 sequences)

SEQ ID NO: 1 - protein, 6 aa, synthetic construct
KRRKLR

SEQ ID NO: 2 - protein, 8 aa, synthetic construct
RERRRKKR

SEQ ID NO: 3 - protein, 8 aa, synthetic construct
THRTRRST

SEQ ID NO: 4 - protein, 8 aa, synthetic construct
NTRSRRSV

SEQ ID NO: 5 - protein, 8 aa, synthetic construct
LRRRRRDA

SEQ ID NO: 6 - protein, 8 aa, synthetic construct
AKRTKRAS

SEQ ID NO: 7 - protein, 8 aa, synthetic construct
TRRFRRSI

SEQ ID NO: 8 - protein, 8 aa, synthetic construct
SRRARRSV

SEQ ID NO: 9 - protein, 8 aa, synthetic construct
SGRSRRSV

SEQ ID NO: 10 - protein, 8 aa, synthetic construct
GSRTRRSV

SEQ ID NO: 11 - protein, 8 aa, synthetic construct
HRREKRSV

SEQ ID NO: 12 - protein, 8 aa, synthetic construct
SGRSKRSV

SEQ ID NO: 13 - protein, 8 aa, synthetic construct
GTRHRRTV

SEQ ID NO: 14 - protein, 8 aa, synthetic construct
LKRRRRDT

SEQ ID NO: 15 - protein, 8 aa, synthetic construct
TNRSKRNL

SEQ ID NO: 16 - protein, 8 aa, synthetic construct
TRKQKRSV

SEQ ID NO: 17 - protein, 8 aa, synthetic construct
YFRRKRSI

SEQ ID NO: 18 - protein, 8 aa, synthetic construct
TRRQKRGL

SEQ ID NO: 19 - protein, 8 aa, synthetic construct
KKREKRGL

SEQ ID NO: 20 - protein, 8 aa, synthetic construct
GRRQRRFI

SEQ ID NO: 21 - protein, 8 aa, synthetic construct
PARSKRGL

SEQ ID NO: 22 - protein, 8 aa, synthetic construct
DPRTKRFF

SEQ ID NO: 23 - protein, 8 aa, synthetic construct
SRRHKRFA

SEQ ID NO: 24 - protein, 8 aa, synthetic construct
SRRHKRFA

SEQ ID NO: 25 - protein, 8 aa, synthetic construct
TRRRRRFA

SEQ ID NO: 26 - protein, 8 aa, synthetic construct
KKRKRRFL

SEQ ID NO: 27 - protein, 8 aa, synthetic construct
NPRQSRFV

SEQ ID NO: 28 - protein, 8 aa, synthetic construct
VQREKRAV

SEQ ID NO: 29 - protein, 8 aa, synthetic construct
GIRRKRSV

SEQ ID NO: 30 - protein, 8 aa, synthetic construct
SNRHKREP

SEQ ID NO: 31 - protein, 8 aa, synthetic construct
SSRRRRDI

SEQ ID NO: 32 - protein, 7 aa, synthetic construct
RVRRKRF

SEQ ID NO: 33 - protein, 7 aa, synthetic construct
RARRKRL

SEQ ID NO: 34 - protein, 7 aa, synthetic construct
KRKRRSL

SEQ ID NO: 35 - protein, 7 aa, synthetic construct
RTKRKRV

SEQ ID NO: 36 - protein, 7 aa, synthetic construct
GRRGKRF

SEQ ID NO: 37 - protein, 7 aa, synthetic construct
SVRRKRT

SEQ ID NO: 38 - protein, 7 aa, synthetic construct
KRRRRRR

SEQ ID NO: 39 - protein, 7 aa, synthetic construct
RRKRKRA

SEQ ID NO: 40 - protein, 7 aa, synthetic construct
QRTRRRS

SEQ ID NO: 41 - protein, 7 aa, synthetic construct
GRRRKRS

SEQ ID NO: 42 - protein, 7 aa, synthetic construct
RSKRKRK

SEQ ID NO: 43 - protein, 7 aa, synthetic construct
KRRKRSR

SEQ ID NO: 44 - protein, 7 aa, synthetic construct
RKRRQRR

SEQ ID NO: 45 - protein, 7 aa, synthetic construct
KRRRKRL

SEQ ID NO: 46 - protein, 7 aa, synthetic construct
RKKRKRK

SEQ ID NO: 47 - protein, 25 aa, synthetic construct
ASYQTQTNSPRERRRKKRSVASQSI

SEQ ID NO: 48 - protein, 5 aa, synthetic construct
GGSGG

SEQ ID NO: 49 - protein, 5 aa, synthetic construct
GGGGS

SEQ ID NO: 50 - protein, 5 aa, synthetic construct
EAAAR

SEQ ID NO: 51 - protein, 5 aa, synthetic construct
GAGAG

SEQ ID NO: 52 - protein, 6 aa, synthetic construct
AKAKAK

SEQ ID NO: 53 - protein, 6 aa, synthetic construct
SGSGSG

SEQ ID NO: 54 - DNA, 795 bp, synthetic construct
  source 1..795: mol_type = other DNA
atggggagtagcaagagcaagcctaaggaccccagccagcgcggaggaagcggaggaagaaagagaagaagaaagctgagatgtacagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagcag

SEQ ID NO: 55 - DNA, 1143 bp, synthetic construct
  source 1..1143: mol_type = other DNA
atggggagtagcaagagcaagcctaaggaccccagccagcgcggaggaagcggaggaagaaagagaagaagaaagctgagatgtacaggcacacgactccccaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgagattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgat

SEQ ID NO: 56 - DNA, 880 bp, synthetic construct
  source 1..880: mol_type = other DNA
atggggagtagcaagagcaagcctaaggaccccagccagcgcggaggaagcggaggaagaaagagaagaagaaagctgagatgtacgcgagctatcagacccagaccaacagcccgcgcgaacgccgccgcaaaaaacgcagcgtggcgagccagagcattacgtacgtacagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagcag

SEQ ID NO: 57 - DNA, 1228 bp, synthetic construct
  source 1..1228: mol_type = other DNA
atggggagtagcaagagcaagcctaaggaccccagccagcgcggaggaagcggaggaagaaagagaagaagaaagctgagatgtacgcgagctatcagacccagaccaacagcccgcgcgaacgccgccgcaaaaaacgcagcgtggcgagccagagcattacgtacgtacaggcacacgactccccaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgagattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgat

SEQ ID NO: 58 - DNA, 5826 bp, synthetic construct
  source 1..5826: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccccaccatggggagtagcaagagcaagcctaaggaccccagccagcgcggaggaagcggaggaagaaagagaagaagaaagctgagatgtacagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagcaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggtccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 59 - DNA, 5826 bp, synthetic construct; identical to SEQ ID NO: 58
  source 1..5826: mol_type = other DNA

SEQ ID NO: 60 - DNA, 5911 bp, synthetic construct
  source 1..5911: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccccaccatggggagtagcaagagcaagcctaaggaccccagccagcgcggaggaagcggaggaagaaagagaagaagaaagctgagatgtacgcgagctatcagacccagaccaacagcccgcgcgaacgccgccgcaaaaaacgcagcgtggcgagccagagcattacgtacgtacagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagcaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggtccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 61 - DNA, 6259 bp, synthetic construct
  source 1..6259: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccccaccatggggagtagcaagagcaagcctaaggaccccagccagcgcggaggaagcggaggaagaaagagaagaagaaagctgagatgtacgcgagctatcagacccagaccaacagcccgcgcgaacgccgccgcaaaaaacgcagcgtggcgagccagagcattacgtacgtacaggcacacgactccccaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgagattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggtccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 62 - DNA, 6507 bp, synthetic construct
  source 1..6507: mol_type = other DNA
gaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcctcgacggatccctcgaggaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcctcgagggatccgtcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacggatcggccgcaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcgggga

SEQ ID NO: 63 - DNA, 81 bp, synthetic construct
  source 1..81: mol_type = other DNA
gtacgcgagctatcagacccagaccaacagcccgcgcgaacgccgccgcaaaaaacgcagcgtggcgagccagagcattac

SEQ ID NO: 64 - DNA, 81 bp, synthetic construct
  source 1..81: mol_type = other DNA
gtacgtaatgctctggctcgccacgctgcgttttttgcggcggcgttcgcgcgggctgttggtctgggtctgatagctcgc

SEQ ID NO: 65 - DNA, 81 bp, synthetic construct; identical to SEQ ID NO: 64
  source 1..81: mol_type = other DNA

SEQ ID NO: 66 - protein, 15 aa, synthetic construct
  VARIANT 8: note = F or A
SGAGLPLXARPGANS

SEQ ID NO: 67 - protein, 6 aa, synthetic construct
GSGGTG

SEQ ID NO: 68 - protein, 12 aa, synthetic construct
GSGGGSGGGGTG

SEQ ID NO: 69 - protein, 22 aa, synthetic construct
GSKESGSVSSEQLAQFRSLDTG

SEQ ID NO: 70 - protein, 18 aa, synthetic construct
KESGSVSSEQLAQFRSLD

SEQ ID NO: 71 - protein, 14 aa, synthetic construct
EGKSSGSGSESKST

SEQ ID NO: 72 - protein, 12 aa, synthetic construct
GSAGSAAGSGEF

SEQ ID NO: 73 - protein, 10 aa, synthetic construct
GSKEAAAKDR

SEQ ID NO: 74 - protein, 4 aa, synthetic construct
  REGION 1..4: note = The entire sequence can be repeated 1-5 times
GGGS

SEQ ID NO: 75 - protein, 12 aa, synthetic construct
  REGION 2..6: note = The sequence of amino acids 2-6 can be repeated 1-4 times
AEAAAKEAAAKA

(19) United States
(12) Patent Application Publication
(10) Pub. No.: US 2026/0355344 A1
(43) Pub. Date: Nov. 12, 2026

(54) EXTRACELLULAR VESICLES FOR DRUG DELIVERY AND USES THEREOF

(71) Applicant: UMC Utrecht Holding B.V., Utrecht (NL)

(72) Inventors: Nazma Fhemaaz Ilahibaks, Utrecht (NL); Joseph Petrus Gerardus Sluijter, Utrecht (NL); Zhiyong Lei, Utrecht (NL)

(21) Appl. No.: 19/488,499

(22) PCT Filed: May 27, 2024
(86) PCT No.: PCT/NL2024/050269

§ 371 Date: Nov. 26, 2025

(30) Foreign Application Priority Data
May 26, 2023 (EP) 23175813.7

(51) Int. Cl. A61K 47/69; A61K 47/54; A61K 47/65; C07K 14/005

(52) U.S. Cl. CPC A61K 47/6901; A61K 47/542; A61K 47/65; C07K 14/005; C12N 2760/20222

(57) ABSTRACT

Extracellular vesicles (EVs) comprising a biologically active compound covalently attached to the internal surface of the EV membrane via at least one endoprotease-cleavable linker, methods for producing the EVs, nucleic acid molecules and host cell useful in the preparation thereof and uses thereof are disclosed.

### CROSS-REFERENCE TO RELATED APPLICATIONS

This application is a national phase entry under 35 U.S.C. § 371 of International Patent Application PCT/NL2024/050269, filed May 27, 2024, designating the United States of America and published as International Patent Publication WO 2024/248610 A1 on Dec. 5, 2024, which claims the benefit under Article 8 of the Patent Cooperation Treaty of European Patent Application Serial No. 23175813.7, filed May 26, 2023.

### STATEMENT REGARDING ELECTRONIC FILING OF A SEQUENCE LISTING

A sequence listing conforming to the requirements of WIPO Standard ST.26 is incorporated herein by reference in its entirety. The sequence listing is submitted as an XML file entitled “ISALFSEQLXML.26.xml,” created on Nov. 25, 2025, and having a size of 99.2 KB.

### TECHNICAL FIELD

The disclosure relates to the field of drug delivery, in particular, to extracellular vesicles comprising biologically active compounds as drug delivery vehicles, particularly extracellular vesicles comprising biologically active compounds, such as small molecule drugs, nucleic acids, proteins, and peptides, as drug delivery vehicles for applications in cardiovascular therapy, cancer therapy, immunotherapy, regenerative medicine, and gene therapy.

### BACKGROUND

Extracellular vesicles (EVs) show potential as drug delivery vehicle for therapeutic compounds, including proteins. Direct cytosolic delivery of functional compounds, in particular larger compounds such as proteins, remains a key issue for therapies targeting intracellular targets. Intracellular delivery requires therapeutic compounds to be trafficked across the cellular membrane and escape the endosome to be released into the cytosol. EVs address this problem because they have an intrinsic ability to shuttle bioactive cargo between cells, evoking phenotypic changes upon delivery to target cells.

EVs are natural cell-derived nanovesicles consisting of a lipid bilayer, facilitating cell-to-cell communications by delivering macromolecules to target cells, including lipids, nucleic acids, and proteins. EVs are secreted by all cell types in the body and have been linked to (patho) physiological processes. In contrast to conventional drug delivery vehicles, such as liposomes and lipid nanoparticles, EVs possess many ideal features as a drug delivery system, including their ability to cross natural barriers, protect biological payloads, their non-immunogenic nature, and their potential for targeted delivery. However, despite the promising attributes, loading target proteins inside EVs and unloading them upon delivery to target cells remains challenging.

Drug loading of EVs is for instance described in WO 2020/191377, which describes loading of biologically active molecules at the external membrane of EVs. The molecules are conjugated to the EVs after EV isolation via a maleimide moiety in the presence of a reducing agent.

Compounds of interest can further be loaded inside EVs through either post-EV isolation (exogenous) or pre-EV isolation (endogenous) methods. Post-EV isolation loading strategies incorporate compounds inside purified EVs through sonication, electroporation, co-incubation, saponin treatment, extrusion, or freeze-thaw cycles. The major drawback of these physical or chemical modification methods is that they could potentially harm the integrity of both the therapeutic compound of, in particular, proteins, and the EV carrier. In contrast, endogenous loading methods, i.e., loading during EV biogenesis, rely on strategic engineering methods to load targeting proteins or peptide-containing compounds inside EVs.

Covalent lipid modifications have been investigated as endogenous protein loading strategies. Among those methods are palmitoylation and N-myristoylation, where the co-/post-translational modification leads to an irreversible attachment of the palmitic acid to cysteine or myristoyl group to the N-terminus of a target protein, respectively (Whitley et al. 2022; Corso et al. 2019). However, these endogenous EV protein-loading strategies have limited cytosolic delivery efficiency because the covalent anchor does not allow the target protein to disassociate from the EVs' lumen and a significant amount of cargo proteins delivered by EVs are destined for lysosomal degradation after uptake in recipient cells (Heusermann et al. 2016).

Despite these methods to anchor compounds to or inside the EVs, there remains a need for versatile and effective strategies that combine and allow for efficient EV loading and delivery of active compounds inside target cells.

### BRIEF SUMMARY

Provided are engineered EVs that overcome one or more of the disadvantages of current EV-based strategies.

The disclosure, therefore, provides an extracellular vesicle (EV) comprising a biologically active compound covalently attached to the internal surface of the EV membrane via at least one endoprotease-cleavable linker.

In a further aspect, the disclosure provides a pharmaceutical composition comprising the EV or nucleic acid molecule, or one or more nucleic acid sequences according to the disclosure and a pharmaceutically acceptable carrier.

In a further aspect, the disclosure provides a method for producing EVs according to the disclosure comprising:
introducing a nucleic acid molecule comprising an oligonucleotide encoding the biologically active compound, the at least one endoprotease-cleavable linker and an attachment moiety for covalent attachment to an EV membrane into a mammalian host cell;
allowing the host cell to produce EVs; and
isolating the produced EVs.

In a further aspect, the disclosure provides a nucleic acid molecule or nucleic acid sequence comprising an oligonucleotide encoding a biologically active compound, in particular a protein, polypeptide or peptide, at least one endoprotease-cleavable linker and an attachment moiety for covalent attachment to an EV membrane.

In a further aspect, the disclosure provides one or more nucleic acid sequences encoding a biologically active compound, in particular a protein, polypeptide or peptide, an oligonucleotide encoding at least one endoprotease-cleavable linker and an oligonucleotide encoding an attachment moiety for covalent attachment to an EV membrane.

In a further aspect, the disclosure provides a mammalian host cell provided with a nucleic acid molecule comprising an oligonucleotide encoding a biologically active compound, at least one endoprotease-cleavable linker and an attachment moiety for covalent attachment of the biologically active compound to an EV membrane into a mammalian host cell.

In a further aspect, the disclosure provides an EV, nucleic acid molecule or sequence, one or more nucleic acid sequences or pharmaceutical composition according to the disclosure for use as a medicament or diagnostic.

In a further aspect, the disclosure provides an EV, nucleic acid molecule or sequence, one or more nucleic acid sequences or pharmaceutical composition according to the disclosure for use in treatment, prevention or diagnosis of disease.

In a further aspect, the disclosure provides a method of treatment or prevention of a disease in an individual in need thereof, comprising administering the EV, nucleic acid molecule or sequence, one or more nucleic acid sequences or pharmaceutical composition according to the disclosure to the individual.

In a further aspect, the disclosure provides a use of an EV, nucleic acid molecule or sequence, one or more nucleic acid sequences according to the disclosure for the preparation of a medicament, in particular for treatment of a disease.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, the additional component(s) not altering the unique characteristic of the disclosure.

The articles “a” and “an” are used herein to refer to one or to more than one (i.e., to at least one) of the grammatical object of the article. By way of example, “an element” means one element or more than one element.

The word “approximately” or “about” when used in association with a numerical value (e.g., approximately 10, about 10) preferably means that the value may be the given value (e.g., 10), plus or minus 5% of the value (e.g., 10, plus or minus 5%), preferably plus or minus 1% of the value.

The use of the alternative (e.g., “or”) should be understood to mean either one, both, or any combination thereof of the alternatives.

As used herein, the term “prevention” refers to precluding or delaying the onset of a disease or condition and/or the appearance of clinical symptoms of the disease or condition in a subject that does not yet experience clinical symptoms of the disease.

The term “treatment” refers to inhibiting the disease or disorder, i.e., halting or reducing its development or at least one clinical symptom of the disease or disorder, and/or to relieving symptoms of the disease or condition. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

As used herein, the term “subject” encompasses humans and animals, preferably mammals. Preferably, a subject is a mammal, more preferably a human.

The terms “polypeptide,” “peptide,” and “protein” are used interchangeably herein and refer to compounds comprising amino acids joined via peptide bonds. The protein, peptide or polypeptide can be naturally occurring, such as a biologically active protein, e.g., antibody, cytokine, growth factor, plasma protein, etc., or a synthetic protein, peptide or polypeptide, e.g., an engineered protein, peptide or polypeptide, such as a variant or part of a biologically active protein, e.g., antibody, cytokine, growth factor, plasma protein, etc. The term “therapeutic protein” is well known in the art and refers to a protein or (poly) peptide that has a therapeutic or prophylactic effect when administered to a subject.

As used herein with respect to the amino acids sequence of a polypeptide, the terms “N-terminal” and “C-terminal” refer to relative positions in the amino acid sequence of the polypeptide toward the N-terminus and the C-terminus, respectively. “N-terminus” and “C-terminus” refer to the extreme amino and carboxyl ends of the polypeptide, respectively. “Immediately N-terminal” and “immediately C-terminal” refers to a position of a first amino acid sequence relative to a second amino acid sequence where the first and second amino acid sequences are covalently bound to provide a contiguous amino acid sequence.

In amino acid sequences as defined herein amino acids are denoted by single-letter symbols. These single-letter symbols, as well as three-letter symbols, are well known to the person skilled in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Leu) is leucine, M (Met) is methionine, N (Asn) is asparagine, P (Pro) is proline, Q (Gln) is glutamine, R (Arg) is arginine, S (Ser) is serine, T (Thr) is threonine, V (Val) is valine, W (Trp) is tryptophan, Y (Tyr) is tyrosine.

As used herein, a nucleic acid molecule or nucleic acid sequence(s) of the disclosure comprises a chain of nucleotides of any length, preferably DNA and/or RNA, including mRNA. In other embodiments, a nucleic acid molecule or nucleic acid sequence of the disclosure comprises other kinds of nucleic acid structures such as for instance a DNA/RNA helix, peptide nucleic acid (PNA), locked nucleic acid (LNA) and/or a ribozyme. The term nucleic acid molecule includes recombinant and synthetic nucleic acid molecules.

The percentage of identity of an amino acid sequence or nucleic acid sequence, or the term “% sequence identity,” is defined herein as the percentage of residues of the full length of an amino acid sequence or nucleic acid sequence that is identical with the residues in a reference amino acid sequence or nucleic acid sequence after aligning the two sequences to achieve the maximum percent identity. As used herein, sequence identity is calculated on the basis of consecutive amino acids of the subject amino acid sequence. Methods and computer programs for the alignment are well known in the art, for example, “Align 2.” Programs for determining nucleotide sequence identity are also well known in the art, for example, the BESTFIT, FASTA and GAP programs. These programs are readily utilized with the default parameters recommended by the manufacturer.

A new EV-based platform technology for the loading and unloading of biologically active compounds of interest in and from EVs has been successfully developed. By expressing covalently anchored linkers containing endoprotease-cleavable sites fused to a protein or (poly) peptide of interest in donor cells, endogenous protein or (poly) peptide loading inside EVs is stimulated. Upon EV-mediated protein or (poly) peptide delivery, unloading of the protein or (poly) peptide is facilitated by endoprotease-cleavable sites within the linker region. FIG. 1 schematically shows the technology of the disclosure. Expression of the constructs of the disclosure (in the Examples herein also referred to as VINCI constructs) inside donor cells increased loading of model protein Cre recombinase within EVs. Moreover, integrating an endoprotease cleavage site in the linker region promoted protein unloading in recipient cells.

Moreover, the EV technology of this disclosure provides a simple and straight-forward process to prepare the drug-loaded EVs of interest using recombinant engineering methods to load proteins or peptide-containing compounds inside EVs. In particular, donor cells that produce the EVs are provided with a polynucleotide encoding the protein or (poly) peptide of interest, attached via one or more endoprotease-cleavable linker to a lipid modification sequence. After appropriate culturing, EVs loaded with the protein or (poly) peptide can be isolated. Such process avoids the need for modification of the EVs after isolation, such as conjugating the active compound to the isolated EVs and dedicated treatment steps to release the covalently attached protein or (poly) peptide from the EV membrane.

The Examples herein describe the development of the EV platform technology wherein model proteins GFP and Cre recombinase are covalently attached by N-myristylation, palmitoylation or a combination thereof to the EV membrane internal surface via one or two furin-cleavable linkers. It is shown that with this strategy the proteins are successfully anchored to the cell membrane and efficiently incorporated into EVs. It is, however, emphasized that the EV technology of this disclosure can be tuned with biologically active compounds that target a wide variety of intracellular targets, thereby providing a high therapeutic potential for previously considered “undruggable” targets. The EV platform technology of the disclosure thus is independent of the specific compound, in particular protein or (poly) peptide, that is used loaded into the EVs and has the demonstrated advantages independently thereof.

In a first aspect, the disclosure therefore provides an extracellular vesicle (EV) comprising a biologically active compound covalently attached to the internal surface of the EV membrane via at least one endoprotease-cleavable linker.

As used herein, the term “extracellular vesicle,” abbreviated as “EV,” refers to a cell-derived vesicle having a lipid bilayer membrane that encloses an internal space. EVs typically range in size from approximately 20 to 1000 nm. They are formed through the outward budding of the cell membrane, giving rise to microvesicles that are released to the external environment, or through the inward budding of the cellular membrane, giving multivesicular bodies that in turn release vesicles upon fusion with the plasma membrane (exosomes). Extracellular vesicles can be prepared by culturing appropriate producer cells in a suitable medium and harvesting extracellular vesicles. The extracellular vesicle according to the disclosure can be a microvesicle or an exosome. In preferred embodiments, the extracellular vesicle is an exosome. As used herein, an exosome preferably refers to an EV having a diameter of 20-300 nm.

The biologically active compound is covalently attached to the internal surface of the EV membrane. As used herein, “EV membrane” refers to the lipid bilayer membrane that separates the internal space of the EV from the external environment. The lipid bilayer membrane protects the material that is encapsulated in the EV, including proteins, nucleic acids, lipids and metabolites, from the extracellular environment. The internal space of the EV is also referred to as the lumen. The “internal surface” of the EV membrane refers to the luminal side of the membrane. Biologically active compounds that are attached to the internal surface of the EV membrane are thus located in the internal space or lumen of the EV.

“Covalent attachment” has its ordinary meaning that is well known in the art and refers to a chemical bond formed when electrons are shared between two atoms. Covalent attachment can be achieved, for example, by peptide bonds or chemical cross-linking. In this disclosure, covalent attachment is preferably achieved by chemical cross-linking. Covalent attachment ensures that when loading biologically active compounds during EV biogenesis, high amounts of biologically active compounds are coupled to the cell membrane of the producer cell and are included in the EVs.

The biologically active compound can be covalently attached to the internal surface by any method suitable for this purpose and known in the art. In preferred embodiments, the biologically active compound is covalently attached to the EV membrane via fatty acid acylation, prenylation or a glycophosphatidylinositol (GPI) anchor, preferably via fatty acid acylation. Combinations thereof are also possible. Fatty acid acylation of proteins is the co- or post-translational covalent linkage of an acyl-CoA derived from a fatty acid to an amino acid residue of the substrate protein. Prenylation is a post-translational modification of proteins whereby a hydrophobic group is added to a protein. A GPI anchor refers to the posttranslational modification wherein a glycosylphosphatidylinositol or glycophosphatidylinositol is attached to the C-terminus of a protein. In preferred embodiments, the biologically active compound is thus covalently attached to the EV membrane via fatty acid, or prenyl group or a combination thereof, including combinations of more than one fatty acid and/or more than one prenyl group. The fatty acid is preferably a saturated fatty acid, because fatty acids that are involved in protein acylation are mostly saturated fatty acids. Preferred examples of fatty acids include palmitic acid (C16:0), myristic acid (C14:0) and caprylic acid (C8:0). Preferred examples of prenyl groups are farnesyl (C15) and geranylgeranyl (C20) moieties.

Hence, in preferred embodiments, the biologically active compound is covalently attached to the EV membrane by S-acylation, palmitoylation, prenylation, N-myristoylation and/or a GPI anchor. In further preferred embodiments, the biologically active compound is covalently attached to the EV membrane by palmitoylation and/or N-myristoylation.

S-acylation is a reversible posttranslational lipid modification of proteins, whereby a long-chain lipid is enzymatically and covalently attached to a cysteine in a protein. Attachment occurs via a labile thioester linkage and thus S-acylation may be reversible, in contrast to other lipid modifications,

Palmitoylation, that occurs post-translational or co-translational, leads to the covalent attachment of palmitic acid, a 16-carbon fatty acid, to a cysteine residue by a thioester bond (S-palmitoylation) or serine or threonine residue (O-palmitoylation) in a target amino acid sequence. S-palmitoylation is a form of S-acylation, whereby the long-chain lipid is palmitate. The modification is catalyzed by palmitoyltransferases (PATs). PATs are characterized by a conserved DHHC (Asp-His-His-Cys) sequence, that is essential for catalytic activity. 23 PATs exhibiting a DHHC sequence have been identified in mammals. PATs are expressed in many cell types and tissues.

Prenylation is a post-translational modification of proteins whereby a hydrophobic group is added to a protein. Typically, one of two prenyl groups, either a farnesyl moiety or a geranylgeranyl moiety, is added to a C-terminal cysteine residue in the protein. The modification can be catalyzed by three different enzymes, farnesyl transferases, Caax proteases and geranylgeranyl transferases.

N-Myristoylation, that also occurs post-translational or co-translational, is the covalent attachment of a myristoyl group derived from myristic acid to the alpha-amino of an N-terminal glycine residue in a protein via an amide bond. The modification is catalyzed by N-myristoyltransferases (NMT1 and NMT2) in the cell cytoplasm.

A GPI anchor is a posttranslational modification of proteins with a glycolipid. The C-terminus of a GPI-anchored protein is linked through a phosphoethanolamine bridge to a highly conserved core glycan, mannose (α1-2) mannose (α1-6) mannose (α1-4) glucosamine (α1-6) myo-inositol.

A single or multiple anchors, in particular single or multiple lipid anchors, may be present.

In preferred embodiments, the biologically active compound is covalently attached to the internal surface of the EV membrane through N-myristoylation. As demonstrated in the Examples herein, proteins can be successfully anchored to the cellular membrane through the lipid modification of an N-Myristoylation sequence present in the protein and loading of protein within EVs is increased by 1.7-2.4-fold as compared to wild-type protein.

In other preferred embodiments, the biologically active compound is covalently attached to the internal surface of the EV membrane through palmitoylation. As demonstrated in the Examples herein, proteins can be successfully anchored to the cellular membrane through the lipid modification of a palmitoylation sequence present in the protein and loading of protein within EVs is increased as compared to wild-type protein.

In other preferred embodiments, the biologically active compound is covalently attached to the internal surface of the EV membrane through myristoylation and palmitoylation, preferably N-myristoylation and palmitoylation. In particularly preferred embodiments, the biologically active compound is covalently attached to the internal surface of the EV membrane via a myristoylation-palmitoylation-palmitoylation sequence. As demonstrated in the Examples herein, proteins can be successfully anchored to the cellular membrane through the lipid modification of a combination of a N-myristoylation and palmitoylation sequence present in the protein (myristoylation-palmitoylation-palmitoylation; referred to as MysPalm of Nmyri-2×Palm) and loading of protein within EVs is increased as compared to wild-type protein.

The term “biologically active compound” refers to a compound that exerts an activity when administered to a subject. The activity can be any activity, including, but not limited to, a therapeutic or prophylactic activity, a binding activity, a diagnostic activity or a targeting activity. In preferred embodiments, the compound has a therapeutic, prophylactic or diagnostic activity. In further preferred embodiments, the compound has a therapeutic and/or prophylactic activity. The compound is a compound that can be covalently attached to the internal surface of the EV membrane, preferably via fatty acid acylation, prenylation or a GPI anchor, more preferably via fatty acid acylation. In preferred embodiments, the biologically active compound is a compound that can be attached to the internal surface of the EV membrane via S-acylation, palmitoylation, prenylation, N-myristoylation and/or a GPI anchor, more preferably palmitoylation and/or N-myristoylation, more preferably N-myristoylation, palmitoylation or combination of N-myristoylation-palmitoylation-palmitoylation.

As detailed herein above, the EV platform technology of the disclosure is independent of the specific compound and as such any biologically active compound can be incorporated in the EV of the disclosure. Preferred, but non-limiting, examples of biologically active compounds include a small molecule, a protein, a polypeptide, a peptide, an antibody, an oligonucleotide, a lipid, a carbohydrate, a detectable label, such as a fluorescent label, a chemiluminescent label, a radionuclide, etc. In preferred embodiments, the biologically active compound is an oligonucleotide, a protein, a polypeptide or a peptide, more preferably a protein, a polypeptide or a peptide.

In some preferred embodiments, the biologically active compound, preferably peptide, polypeptide or protein, comprises or is modified to comprise at least one endoprotease-cleavable linker and a glycine residue that can react with a myristic acid.

In some preferred embodiments, the biologically active compound, preferably peptide, polypeptide or protein, comprises or is modified to comprise at least one endoprotease-cleavable linker and a cysteine residue or serine or threonine residue that can react with a palmitic acid.

In some preferred embodiments, the biologically active compound, preferably peptide, polypeptide or protein, comprises or is modified to comprise at least one endoprotease-cleavable linker and a cysteine residue that can react with a prenyl group.

In some preferred embodiments, the biologically active compound, preferably peptide, polypeptide or protein, comprises or is modified to comprise at least one endoprotease-cleavable linker and a glycine residue that can react with a myristic acid and/or a palmitic acid.

The EV of the disclosure comprises a plurality of the biologically active compound. The EV may further comprise more than two or more different biologically active compounds.

In the EV of the disclosure, the biologically active compound is covalently attached to the internal surface of the EV membrane via at least one endoprotease-cleavable linker. As used herein, the term “endoprotease” refers to any endogenous protease, also referred to as peptidase, that catalyses the cleavage of an internal bond in a peptide, polypeptide or protein. The endoprotease is preferably endogenous to the target cell type, e.g., a mammalian endoprotease or a human endoprotease. The use of such endoprotease-cleavable linker allows the cleavage of the linker in the target cell after administration of the EVs. As such, the endoprotease-cleavable linker allows for release of the biologically active compound from the EV membrane and, consequently, allows for the biological activity of the compound. Since the target cells of the EVs are, in particular, mammalian cells, more particularly human cells, the endoprotease is preferably a mammalian endoprotease, more preferably a human endoprotease. In preferred embodiments, the endoprotease is further an intracellular endoprotease, preferably a mammalian intracellular endoprotease, more preferably a human intracellular endoprotease. “Intracellular endoprotease” refers to an endoprotease that exerts protease activity intracellularly.

Any linker or sequence that can be cleaved by any endoprotease can be used. Non-liming examples of suitable endoproteases include serine proteases, such as furin, tissue plasminogen activator, activated protein C, coagulation factors, such as factor VIIa, IXa, Xa, XIa, XIIa, thrombin, plasmin, granzymes, subtilases (such as furin; membrane bound transcription factor peptidase, site 1 (MBTPS1); proprotein convertase subtilisin/kexin type 1 (PCSK1), PCSK2, PCSK4, PCSK5, PCSK6, PCSK7, PCSK9, tripeptidyl peptidase II (TPP2)), cathepsins, prolyl endopeptidase, urokinase, matrix metallopeptidases, metalloprotease-related proteins, kallikreins, kallikrein related peptidases, plasma kallikrein, aminopeptidases, chymopasin, prosemin, trypsin, tryptases, acrosin, hepsin, neurotrypsin, ADAMTS peptidases, ADAM peptidases, cysteine proteases, such as calpains (e.g., calpain-1, calpain-2 and calpain-3), dipeptidyl-peptidases, cathepsins (e.g., B, C. H, K, L and S), caspases (e.g., caspase-1, -2, -3, -6, -7, -8, -9 and -10), ubiquitin-specific proteases (USPs, such as USP7, USP14 and USP9X), legumain and papain, pepsin, gastricsin, presenilin, signal peptide peptidase-like protein, calpamodulin, ubiquitinyl hydrolases, ubiquitin-specific peptidases, SENP peptidases, carboxypeptidases, metallocarboxypeptidases, dihydropyrimidinase related proteins, tryptase beta, complement components, matriptase, gamma-glutamyltransferase, gamma-glutamyltransferase like proteins, separase, proprotein convertases, caspases and combinations thereof.

In preferred embodiments, the endoprotease is an intracellular serine protease or an intracellular cysteine protease and the at least one cleavable linker is at least one intracellular serine protease and/or intracellular protease-cleavable linker.

Preferred examples of intracellular serine proteases are subtilases, including furin, MBTPS1, PCSK1, PCSK2, PCSK4, PCSK5, PCSK6, PCSK7, PCSK9, TTP2, prolyl endopeptidase, and cathepsins, including cathepsin B, cathepsin C, cathepsin H, cathepsin K, cathepsin L and cathepsin S. Preferred examples of intracellular cysteine proteases are caspases, including caspase-1, caspase-2, caspase-3, caspase-6, caspase-7, caspase-8, caspase-9 and caspase-10), cathepsins, including cathepsin B, cathepsin C, cathepsin H, cathepsin K, cathepsin L and cathepsin S, calpains, including calpain-1, calpain-2 and calpain-3, Ubiquitin-Specific Proteases, including USP7, USP14 and USP9X and legumain. Hence, in preferred embodiments, the endoprotease is selected from the group consisting of subtilases, including furin, MBTPS1, PCSK1, PCSK2, PCSK4, PCSK5, PCSK6, PCSK7, PCSK9, TTP2, prolyl endopeptidase, caspases, including caspase-1, caspase-2, caspase-3, caspase-6, caspase-7, caspase-8, caspase-9 and caspase-10), cathepsins, including cathepsin B, cathepsin C, cathepsin H, cathepsin K, cathepsin L and cathepsin S, calpains, including calpain-1, calpain-2 and calpain-3, Ubiquitin-Specific Proteases, including USP7, USP14 and USP9X, legumain and combinations thereof, and the at least one cleavable linker is at least one subtilase, prolyl endopeptidase, caspases, cathepsins, calpain, Ubiquitin-Specific Protease, and/or legumain-cleavable linker.

In preferred embodiments, the endoprotease is selected from the group consisting of subtilases, calpains and combinations thereof and the at least one cleavable linker is at least one subtilase- and/or calpain-cleavable linker.

In preferred embodiments, the endoprotease is selected from the group consisting of furin, calpain, and combinations thereof and the at least one cleavable linker is at least one furin- and/or calpain-cleavable linker.

In preferred embodiments, the endoprotease is furin and the at least one cleavable linker is at least one furin-cleavable linker.

In other preferred embodiments, the endoprotease is calpain and the at least one cleavable linker is at least one calpain-cleavable linker.

In other preferred embodiments, the endoprotease is calpain and furin, and the at least one cleavable linker is at least one furin-cleavable linker and at least one calpain-cleavable linker.

The biologically active compound is covalently attached to the internal surface of the EV membrane via at least one endoprotease-cleavable linker. The at least one endoprotease-cleavable linker is preferably between one and five endoprotease-cleavable linkers, more preferably between one and three, more preferably one or two. As demonstrated in the Examples herein, the use of a single- and double-cleavable linker was successful in achieving cleavage of a model compound that was attached to the internal surface of the EV membrane.

If more than one endoprotease-cleavable linker is used the linker can be the same of different. Further, the endoprotease that can cleave the linker can be the same of different. For example, if two endoprotease-cleavable linkers are present, these can be the same or a different furin-cleavable linker, or one can be, e.g., a furin-cleavable linker and the other, e.g., a kallikrein- or calpain-cleavable linker.

In preferred embodiments, the at least one cleavable linker is one, two, three, four or five endoprotease-cleavable linker, preferably one, two or three endoprotease-cleavable linker, more preferably one or two endoprotease-cleavable linkers.

In preferred embodiments, the at least one cleavable linker is one, two, three, four or five subtilase, prolyl endopeptidase, caspases, cathepsins, calpain, ubiquitin-specific protease, and/or legumain-cleavable linkers, preferably one, two or three subtilase, prolyl endopeptidase, caspases, cathepsins, calpain, ubiquitin-specific protease, and/or legumain-cleavable linkers, more preferably one or two subtilase, prolyl endopeptidase, caspases, cathepsins, calpain, ubiquitin-specific protease, and/or legumain-cleavable linkers.

In preferred embodiments, the at least one cleavable linker is one, two, three, four or five subtilase and/or calpain, preferably furin- and/or calpain-cleavable linkers, preferably one, two or three subtilase and/or calpain, preferably furin- and/or calpain-cleavable linkers, more preferably one or two subtilase and/or calpain, preferably furin- and/or calpain-cleavable linkers.

Any known endoprotease, preferably intracellular endoprotease, more preferably subtilase, prolyl endopeptidase, caspases, cathepsins, calpain, Ubiquitin-Specific Protease, and/or legumain, more preferably furin- and/or calpain-cleavable linker can be used in an EV according to the disclosure and a skilled person is well capable of selecting at least one suitable endoprotease-cleavable linker. In preferred embodiments, the at least one linker is composed of an amino acid chain, i.e., the endoprotease-cleavable linker is preferably an endoprotease-cleavable peptide linker. The length of the endoprotease-cleavable linker can be adjusted to optimize the delivery efficiency of the specific biologically active molecule, in particular protein polypeptide or peptide and preferably ranges from 2-50 amino acids in length, more preferably from 2-25 amino acids, more preferably from 2-20 amino acids, more preferably from 2-15 amino acids, more preferably from 2-12 amino acids, such as 2, 3, 4, 5, 6, 7, 8, 9, 10, 11 or 12 amino acids. In further preferred embodiments, the endoprotease-cleavable peptide linker has a length of between 5 and 10 amino acids, such as 5, 6, 7, 8, 9 or 10 amino acids. In some embodiments, the endoprotease-cleavable peptide linker has a length of 6, 7 or 8 amino acids.

If the at least one endoprotease-cleavable linker is one or more furin-cleavable linkers, the linker or linkers can be any known furin-cleavable linker. The canonical furin cleavage motif is typically represented as RX (K/R) RI, where X can be any amino acid and the arrow (1) indicates the cleavage site. Hence, in a preferred embodiment, the one or more furin-cleavable linkers comprise or consist of the sequence RX (K/R)R, where R is arginine, K is lysine and X can be any amino acid. A furin cleavage site can be of any known mammalian, viral and bacterial substrates, such as human coronavirus spike protein furin cleavage sites and influenza HA furin cleavage sites. Non-limiting examples of suitable furin-cleavable linkers include linkers comprising or consisting of the sequence KRRKLR (SEQ ID NO:1), the H5N1 avian influenza haemagglutinin cleavage site containing a string of basic amino acids (H5CS; sequence:

(SEQ ID NO: 2))
RERRRKKR,
(SEQ ID NO: 3)
THRTRRST,
(SEQ ID NO: 4)
NTRSRRSV,
(SEQ ID NO: 5)
LRRRRRDA,
(SEQ ID NO: 6)
AKRTKRAS,
(SEQ ID NO: 7)
TRRFRRSI,
(SEQ ID NO: 8)
SRRARRSV,
(SEQ ID NO: 9)
SGRSRRSV,
(SEQ ID NO: 10)
GSRTRRSV,
(SEQ ID NO: 11)
HRREKRSV,
(SEQ ID NO: 12)
SGRSKRSV,
(SEQ ID NO: 13)
GTRHRRTV,
(SEQ ID NO: 14)
LKRRRRDT,
(SEQ ID NO: 15)
TNRSKRNL,
(SEQ ID NO: 16)
TRKQKRSV,
(SEQ ID NO: 17)
YFRRKRSI,
(SEQ ID NO: 18)
TRRQKRGL,
(SEQ ID NO: 19)
KKREKRGL,
(SEQ ID NO: 21)
PARSKRGL,
(SEQ ID NO: 20)
GRRQRRFI,
(SEQ ID NO: 22)
DPRTKRFF,
(SEQ ID NO: 23)
SRRHKRFA,
(SEQ ID NO: 24)
SRRHKRFA,
(SEQ ID NO: 25)
TRRRRRFA,
(SEQ ID NO: 26)
KKRKRRFL,
(SEQ ID NO: 27)
NPRQSRFV,
(SEQ ID NO: 28)
VQREKRAV,
(SEQ ID NO: 29)
GIRRKRSV,
(SEQ ID NO: 30)
SNRHKREP,
(SEQ ID NO: 31)
SSRRRRDI,
(SEQ ID NO: 32)
RVRRKRF,
(SEQ ID NO: 33)
RARRKRL,
(SEQ ID NO: 34)
KRKRRSL,
(SEQ ID NO: 35)
RTKRKRV,
(SEQ ID NO: 36)
GRRGKRF,
(SEQ ID NO: 37)
SVRRKRT,
(SEQ ID NO: 38)
KRRRRRR,
(SEQ ID NO: 39)
RRKRKRA,
(SEQ ID NO: 40)
QRTRRRS,
(SEQ ID NO: 41)
GRRRKRS,
(SEQ ID NO: 42)
RSKRKRK,
(SEQ ID NO: 43)
KRRKRSR,
(SEQ ID NO: 44)
RKRRQRR,
(SEQ ID NO: 45)
KRRRKRL,
and
(SEQ ID NO: 46)
RKKRKRK.

In preferred embodiments, the furin cleavage site comprises or consists of the sequence KRRKLR (SEQ ID NO:1), RERRRKKR (SEQ ID NO:2) or ASYQTQTNSPRERRRKKRSVASQSI (SEQ ID NO: 47).

An example of a suitable calpain-cleavable linker is a linker comprising or consisting of the sequence SGAGLPLF/AARPGANS. However, calpain-cleavable sequences are known in the art and a skilled person is well able to select alternative calpain-cleavable sequences.

The EV may comprise a linking sequence located between the at least one endoprotease-cleavable linker and the site of covalent attachment to the internal surface of the EV membrane. Such linker may provide for flexibility in the molecule that is attached to the EV membrane internal surface, but may also provide charge and hydrophobicity, which may be desired depending on the nature and identity of the biologically active compound. Suitable and optimal linker sequences depend on the specific application, biologically active compound and target cells. Peptide linker for attachments of, e.g., proteins and polypeptides to a cell membrane are widely used and a skilled person is well capable of selecting suitable linkers. Preferably the linking sequence is an amino acid sequence, i.e., a peptide linking sequence, preferably consisting of between 2 and 30 amino acids, more preferably between 3 and 30 amino acid, more preferably between 4 and 30 amino acids, more preferably between 5 and 30 amino acids, more preferably between 6 and 30 amino acids, such as between 10 and 30 amino acids or between 2 and 10 amino acids. In some preferred embodiments, the linking sequences consists of between 2 and 10 amino acids, such as 2, 3, 4, 5, 6, 7, 8, 9 or 10 amino acids. In preferred embodiments, the linking sequence consists of between 2 and 7 amino acids, more preferably of between 3 and 6 amino acids. In other preferred embodiments, the linking sequence has a length of between 10 and 30 amino acids, such as 10, 11, 12, 13, 14, 15, 16, 17, 18, 19, 20, 21, 22, 23, 24 or 25 amino acids. In preferred embodiments, the linking sequence has a length of between 5 and 25 amino acids. In principle any amino acid sequence of such length can be used, as the sequence does not have any functional activity or property. Preferred, but non-limiting examples are sequences comprising or consisting of the sequence GGSGG (SEQ ID NO:48), GGGGS (SEQ ID NO:49), EAAAR (SEQ ID NO:50), GAGAG (SEQ ID NO:51), AKAKAK (SEQ ID NO:52), SGSGSG (SEQ ID NO:53), GSGGTG, AAY, TAT, GSGGGSGGGGTG, GSKESGSVSSEQLAQFRSLDTG, KESGSVSSEQLAQFRSLD, EGKSSGSGSESKST, GSAGSAAGSGEF, GSKEAAAKDR, (GGGS)ₙ, wherein n=1-5, a proline rich linker (AP)ₙ, wherein n=2-30, and a helical linker A(EAAAK)ₙA, wherein n=2-5. GGSGG (SEQ ID NO:48) and GGGGS (SEQ ID NO:49) are linker composed of glycine (G) residues, which provide flexibility and allows for attachment to the membrane without interfering with the attached biomolecule. EAAAR (SEQ ID NO:50) is a negatively charged linker composed of glutamic acid (E) and arginine (R) residues that has been used for attachment of proteins and peptides to the cell membrane. GAGAG (SEQ ID NO:51) is a short, alternating sequence of glycine (G) and alanine (A) residues that provides flexibility and allows for attachment to the membrane without interfering with the attached biomolecule. AKAKAK (SEQ ID NO:52) is a positively charged linker composed of alternating lysine (K) and alanine (A) residues that has been used for attachment of proteins and peptides to the cell membrane. SGSGSG (SEQ ID NO:53) is a short, flexible linker composed of serine(S) and glycine (G) residues that has been used for attachment of peptides and proteins to the cell membrane. AAY is a short, positively charged linker composed of alanine (A) and tyrosine (Y) residues that has been used for attachment of DNA to the cell membrane. TAT is a short, positively charged linker composed of arginine (R) and lysine (K) residues that has been used for attachment of peptides and proteins to the cell membrane.

The EVs of the disclosure may comprise one or more additional compound or compounds. Preferably, such compound is a compound that stimulates or enhances loading of the biologically active compound, in particular protein, polypeptide, or peptide loading, into the EVs and/or release of EV's in target cells. Preferably, such compound is a compound that facilitates the interaction of EVs with target cells and, as a result, enhances their uptake in the target cells. As such, the compound enhances delivery of biologically active compound, in particular protein, polypeptide, or peptide, via the EVs to target cells.

Such compound can be introduced into the EV, expressed on the EV membrane or both. Expression on the EV membrane can be either expression on the external surface of the EV membrane or expression on the internal surface of the EV membrane. In preferred embodiments, the compound is a protein or polypeptide. In further preferred embodiments, the compound or compounds, preferably protein or polypeptide, is expressed on the outer surface of the EV membrane.

In preferred embodiments, the compound is selected from the group consisting of vesicular stomatitis virus G (VSV G) protein or a homolog thereof, such as syncytin-1 and syncytin-2, herpes simplex virus glycoprotein B (HSV gB), rabies viral glycoprotein, influenza virus hemagglutinin (HA), human immunodeficiency virus envelope glycoprotein gp160 (gp160), Caenorhabditis elegans Eff-1 and/or Caenorhabditis elegans Aff-1, or an active part thereof. As used herein, the term “active part” of a compound or protein refers to a part of compound or protein, respectively, that has the same functional activity as the compound or protein, respectively. In the present case, the active part of the recited compounds is a part that stimulates or enhances loading of the biologically active compound, in particular protein, polypeptide, or peptide loading, into the EVs. As used herein, the term “homolog” refers to an equivalent compound or protein that performs the same biological function from another species than the reference species. In particular, a homolog of VSV G protein is an equivalent of VSV G protein from a species other than Vesicular Stomatitis Virus, such as a human homolog. The compound or part thereof is preferably expressed on the outer surface of the EV membrane. In further preferred embodiments, the EV expresses VSV G protein or a homolog thereof, such as syncytin-1 and syncytin-2 on the external surface of the EV membrane. In further preferred embodiments, the EV expresses VSV G protein on the external surface of the EV membrane.

Expression of a compound can for instance be achieved by transfection of the EV producer cell with a nucleic acid molecule comprising a nucleic acid sequence encoding the compound, such as a plasmid. For instance, after transfection of a VSV-G encoding plasmid in the producer cell, VSV-G is expressed automatically on the EVs outer membrane. Expression of VSV-G on the internal surface of EVs is not expected, but, if for some reason VSV-G is found in the internal space or lumen of the EV, it is not detrimental to the EV's function.

Suitable techniques for such preparation are known in the art and described in more detail below and in the Examples herein. In one aspect, the disclosure provides a method for producing extracellular vesicles (EVs) according to the disclosure comprising:
introducing a nucleic acid molecule comprising an oligonucleotide encoding the biologically active compound, the at least one endoprotease-cleavable linker and an attachment moiety for covalent attachment to an EV membrane into a mammalian host cell;
allowing the host cell to produce EVs; and
isolating the produced EVs.

In some preferred embodiments, the EV of the disclosure is prepared by a method comprising:
introducing a nucleic acid molecule comprising an oligonucleotide encoding the biologically active compound, the at least one endoprotease-cleavable linker and an attachment moiety for covalent attachment to an EV membrane into a mammalian host cell;
allowing the host cell to produce EVs; and
isolating the produced EVs.

The term “host cell” as used herein refers to any cell capable of producing extracellular vesicles. In preferred embodiments, the host cell is a cell that is capable of producing a heterologous protein, polypeptide or peptide. The term “host cell” encompasses to any eukaryotic or prokaryotic cell (e.g., bacterial cells such as E. coli, yeast cells, mammalian cells, avian cells, amphibian cells, plant cells, fish cells, and insect cells). Host cells may be in vitro or in vivo, e.g., located in a transgenic animal.

Any cell that is capable of producing EVs can be used as host cell, such cells include cells from a cell line and primary cells, including all adult-derived progenitor and stem cells. Suitable, but non-limiting examples of host cells, include HEK293, HER911, PER.C6, CHO, MSC-1, MCF-7, MDA-MB-231, Huh7, A549, HepG2, THP-1, PC3, B16-F10, C2C12, RAW264.7, A549, NIH-3T3, CT26.WT, EL4, HCT116, SCC-7, PANC-1, 4T1, MC38, U937, Jurkat, K562, Raji, Ramos, dendritic cells, platelets, macrophages, mesenchymal stem cells (MSCs) such as adipose-derived stem cells (ADSCs), bone marrow-derived mesenchymal stem cells (BM-MSCs), placenta-derived mesenchymal stem cells (PD-MSCs), and umbilical cord-derived mesenchymal stem cells (UC-MSCs), induced pluripotent stem cells (iPSCs), cardiac-derived progenitor cells (CPCs), cardiomyocytes, endothelial cells, fibroblasts, Schwann cells, astrocytes, oligodendrocytes, neurons, myoblasts, chondrocytes, tenocytes, hepatocytes, glioblastoma cells, and pancreatic islet cells.

In further preferred embodiments, the host cell is selected from the group consisting of HEK293, HER911, PER.C6®, CHO and MSC-1 cells.

In some preferred embodiments, the host cell is selected from the group consisting of HEK293, HER911, PER.C6®, and MSC-1 cells.

In some preferred embodiments, the host cell is a HEK293 cell.

In some preferred embodiments, the host cell is a human host cell, such as PER.C6.

A host cell in accordance with the disclosure is used to prepare EVs as disclosed herein. A host cell is also referred to as a “producer cell”. In some preferred embodiments, the host cell has a reduced activity or expression of the endoprotease for which a cleavable linker is used to attach the biologically active compound to the internal surface of the EV membrane. This provides the advantage that cleavage of the endoprotease-cleavable linker is reduced or avoided during production of the EVs by the host cell and cleavage of the endoprotease-cleavable linker mainly occurs in the target cell after administration of the EVs of the disclosure. As such, the introduction of biologically active compounds in the EVs, i.e., attached via the endoprotease-cleavable linker, is increased. It also enhances the stability of the endoprotease-cleavable linker and consequently the EVs comprising the biologically active compound according to the disclosure. As such, the use of endoprotease knock-out host cell and/or an endoprotease inhibitor enhances the efficiency of the EV drug delivery system of the disclosure.

Reduced activity or expression of such endoprotease can be achieved using any method known in the art for reducing activity or expression of a protein, in particular an endoprotease.

An example thereof includes the use of engineered host cells that have a reduced expression of the endoprotease, including endoprotease knock-out cells. Such host cell can be prepared by any known method for reducing expression and/or knock-out of the polynucleotide and/or gene encoding the endoprotease, e.g., by RNA interference, and gene editing using genetic editing tools such as zinc finger nucleases, transcription activator-like effectors (TALEN) and CRISPR-Cas technology.

As another example, the host cell can be cultured in the presence of an inhibitor of the endoprotease, such as a small molecule inhibitor of the endoprotease. For example, a method of the disclosure for producing EVs according to the disclosure is performed in the presence of an endoprotease inhibitor. In particular, at least the step of allowing the host cell to produce EVs is performed in the presence of an endoprotease inhibitor. This can for instance be achieved by using culture medium comprising the endoprotease inhibitor and comprising the host cells when performing a method of the disclosure, in particular, for the step of allowing the host cell to produce EVs.

Hence, in a preferred embodiment, the host cell is a protease, in particular, intracellular endoprotease, knock-out host cell and/or the method is performed in the presence of an inhibitor of the protease, in particular intracellular endoprotease. In preferred embodiments, the host cell is a serine protease, such as furin, tissue plasminogen activator, activated protein C, coagulation factor, such as factor VIIa, IXa, Xa, XIa, XIIa, thrombin, plasmin, granzyme, cathepsin, urokinase, matrix metallopeptidase, metalloprotease-related protein, kallikrein, kallikrein related peptidase, plasma kallikrein, aminopeptidase, chymopasin, prosemin, trypsin, tryptases, acrosin, hepsin, neurotrypsin, ADAMTS peptidase, ADAM peptidase, calpain, dipeptidyl-peptidase, pepsin, gastricsin, presenilin, signal peptide peptidase-like protein, calpamodulin, ubiquitinyl hydrolases, ubiquitin-specific peptidase, SENP peptidase, carboxypeptidase, metallocarboxypeptidase, dihydropyrimidinase related protein, tryptase beta, complement component, matriptase gamma-glutamyltransferase, gamma-glutamyltransferase like proteins, separase, proprotein convertase or caspase and combinations thereof. In preferred embodiments, the host cell is a subtilase, prolyl endopeptidase, caspases, cathepsins, calpain, Ubiquitin-Specific Protease, and/or legumain knock-out host cell. In further preferred embodiments, the host cell is a subtilase and/or calpain knock-out, more preferably a furin and/or calpain knock-out, more preferably furin knock-out, host cell.

In other preferred embodiments, the method is performed in the presence of an inhibitor of subtilase, prolyl endopeptidase, caspases, cathepsins, calpain, Ubiquitin-Specific Protease, and/or legumain, more preferably an inhibitor of subtilase and/or calpain, more preferably an inhibitor of furin and/or calpain, more preferably an inhibitor of furin.

In preferred embodiments, the host cell is a furin knock-out host cell.

In other preferred embodiments, the host cell is cultured in the presence of a furin inhibitor and/or the method, or at least the step of allowing the host cell to produce EVs, is performed in the presence of a furin inhibitor.

Suitable ways to deliver the biologically active compound to the host cell is dependent on the type and identity of the biologically active compound. In preferred embodiments, the biologically active compound is a protein, polypeptide or peptide and the host cell is provided with a nucleic acid molecule that encodes the protein, polypeptide or peptide, such that the host cell produces the protein, polypeptide or peptide. The host cell is therefore preferably a cell that is capable of producing proteins, polypeptides and peptides. Also provided is a nucleic acid molecule or nucleic acid sequence comprising an oligonucleotide encoding the biologically active compound as disclosed herein, in particular, a protein, polypeptide or peptide, at least one endoprotease-cleavable linker as disclosed herein and an attachment moiety for covalent attachment to an EV membrane as disclosed herein. Also provided are one or more nucleic acid sequences encoding a biologically active compound, in particular a protein, polypeptide or peptide, an oligonucleotide encoding at least one endoprotease-cleavable linker and an oligonucleotide encoding an attachment moiety for covalent attachment to an EV membrane.

The oligonucleotide encoding the biologically active compound, in particular a protein, polypeptide or peptide, at least one endoprotease-cleavable linker and attachment moiety comprises an oligonucleotide sequence encoding the biologically active compound, an oligonucleotide sequence encoding the at least one endoprotease-cleavable linker and the attachment moiety. The oligonucleotide sequence encoding the at least one endoprotease-cleavable linker is located between the oligonucleotide sequence encoding the biologically active compound and the oligonucleotide encoding the attachment moiety. The expression product of the oligonucleotide comprises the biologically active compound coupled to the attachment moiety via the at least one endoprotease-cleavable linker. Optionally one or more linking sequence may further be present in the expression product, located between the attachment moiety and the at least one endoprotease-cleavable linker and/or between the at least one endoprotease-cleavable linker and the biologically active compound.

The attachment moiety is for covalent attachment of the biologically active compound via the at least one endoprotease-cleavable linker to the EV membrane, in particular the internal surface of the EV membrane. The oligonucleotide sequence encoding the attachment moiety preferably encodes an amino acid sequence for fatty acid acylation, prenylation and/or a GPI anchor, more preferably for S-acylation, palmitoylation, prenylation, N-myristoylation and/or a GPI anchor, more preferably N-myristoylation, palmitoylation and/or prenylation, more preferably N-myristoylation and/or palmitoylation. Amino acid sequences or sites for S-acylation, palmitoylation, prenylation, N-myristoylation or introduction of a GPI anchor are known to and can be appropriately selected by a person skilled in the art. Examples of a consensus sequence or recognition signal for prenylation is CaaX (wherein “a” is an aliphatic amino acid and “X” is any amino acid, typically M, Q, S, T, or A for farnesyl and L or I for geranylgeranyl), -CC or -CXC (wherein “X” is a variable amino acid, typically M, Q, S, T, or A for farnesyl and L or I for geranylgeranyl). An example of a consensus sequence or recognition signal for N-myristoylation is MGxxxS/T/C, wherein “x” is any amino acid except proline (P), aromatic or charged residues in position x3. Palmitoylation sites contain a cysteine or serine or threonine that is often adjacent to a myristoylation and/or prenylation site, have basic or hydrophobic surrounding amino acids and are frequently located in the cytoplasmic regions flanking transmembrane domains or within transmembrane domain.

The oligonucleotide comprised in a nucleic acid molecule or nucleic acid sequence(s) of the disclosure or used in the disclosure may further comprise a nucleotide sequence encoding a linking sequence as disclosed herein, located between the nucleotide sequence encoding the at least one endoprotease-cleavable linker and the nucleotide sequence encoding the attachment moiety.

A nucleic acid molecule or nucleic acid sequence(s) of the disclosure or used in the disclosure may further comprise one or more regulatory sequences to direct expression of one or more of the nucleotide sequences present on the nucleic acid molecule. Examples include a promoter, an enhancer, a transcription termination signal, and a polyadenylation sequence.

In preferred embodiments, a nucleic acid molecule or nucleic acid sequence(s) of the disclosure comprises a promoter operably linked to the oligonucleotide encoding the biologically active compound, at least one endoprotease-cleavable linker and attachment moiety. A used herein, “operably linked” means that a nucleotide sequence is functionally associated with one or more other nucleotide sequences. For instance, the promoter nucleotide sequence is functionally associated with the nucleotide sequence encoding the biologically active compound, at least one endoprotease-cleavable linker and attachment moiety, such that the promoter sequence influences or directs the expression of the other nucleotide sequences. Various promoters, including synthetic and inducible promoters, may be used to direct expression of nucleotide sequences included in a nucleic acid molecule or nucleic acid sequence(s) of the disclosure. Both cell type specific and ubiquitous promoters can be used. Suitable promoters include a SV40 promoter, a Rous Sarcoma Virus (RSV) promoter, a cytomegalovirus (CMV) promoter or derivatives of any of these promoters.

A method of the disclosure for producing EVs may further comprise providing the host cell with a nucleotide sequence encoding one or more compounds that stimulate or enhance loading of the biologically active compound, in particular protein, polypeptide, or peptide loading, into the EVs and/or release of EV's in target cells. In preferred embodiments, the compound is a protein or polypeptide. In further preferred embodiments, the compound or compounds, preferably protein or polypeptide, is expressed on the outer surface of the EV membrane. In preferred embodiments, the compound is selected from the group consisting of vesicular stomatitis virus G (VSV G) protein or a homolog thereof, such as syncytin-1 and syncytin-2, herpes simplex virus glycoprotein B (HSV gB), rabies viral glycoprotein, influenza virus hemagglutinin (HA), human immunodeficiency virus envelope glycoprotein gp160 (gp160), Caenorhabditis elegans Eff-1 and/or Caenorhabditis elegans Aff-1, or an active part thereof as disclosed herein, and combinations or fusions thereof.

The nucleotide sequence encoding one or more compounds that stimulate or enhance loading of the biologically active compound may be comprised in the nucleic acid molecule encoding a biologically active compound, in particular a protein, polypeptide or peptide, at least one endoprotease-cleavable linker and an attachment moiety of the disclosure. Alternatively, a separate nucleic acid molecule may be used that comprises the nucleotide sequence encoding one or more compounds that stimulate or enhance loading of the biologically active compound.

A nucleic acid molecule may be introduced into the host cell by any method known in the art, such as by transfection, transduction, e.g., lentiviral transduction or retroviral transduction, baculoviral expression system, DNA electroporation, or RNA electroporation. The nucleic acid molecule is either transiently or stably provided to the cell. Methods for transfection, transduction or electroporation of cells with a nucleic acid are known to the skilled person.

Alternatively, EVs according to the disclosure can be produced in vivo, e.g., after administration of, e.g., a nucleic acid molecule or nucleic acid sequence or one or more nucleic acid sequence(s) according to the disclosure to a subject. In particular, one or more nucleic acid sequences can be delivered to an organ, in particular, the liver, of a subject, following which EV's according to the disclosure are produced by the organ, in particular liver.

A nucleic acid molecule or nucleic acid sequence(s) according to the disclosure can be introduced in a subject using any method known in the art.

For example, the nucleic acid molecule or nucleic acid sequence(s) is an expression vector. The vector preferably additionally comprises means for high expression levels such as strong promoters, for example, of viral origin (e.g., human cytomegalovirus) or promoters derived from genes that are highly expressed in a cell such as a mammalian cell. The expression vector can be a viral or non-viral vector. Non-limiting examples of suitable expression vectors include retroviral, adenoviral, adeno-associated viral and herpes simplex viral vectors, non-viral vectors and engineered vectors. Non-viral expression vectors include nude DNA, and nucleic acid molecules or sequences packaged into synthetic or engineered compositions such as liposomes, polymers, (lipid) nanoparticles and molecular conjugates. Methods for the generation of such non-viral expression vectors are well known in the art. As an alternative, a nucleic acid molecule or nucleic acid sequence(s) according to the disclosure may be provided to a subject by gene editing technology, including CRISPR/Cas, zinc-finger nucleases, and transcription activator-like effector nucleases-TALEN.

Also provided is a pharmaceutical composition comprising an EV, nucleic acid molecule or one or more nucleic acid sequences according to the disclosure and at least one pharmaceutically acceptable carrier, diluent and/or excipient. By “pharmaceutically acceptable” it is meant that the carrier, diluent or excipient must be compatible with the other ingredients of the formulation and not deleterious, e.g., toxic, to the recipient thereof. In general, any pharmaceutically suitable additive that does not interfere with the function of the active compounds can be used. A pharmaceutical composition according to the disclosure is preferably suitable for human use.

In a preferred embodiment, the suitable carrier is a solution, for example, saline. The pharmaceutical composition is preferably a formulation for parenteral administration. Formulations for parenteral administration include intraarticular, intracardiac, intramuscular, intraocular, intravenous, intraventricular, intraarterial, intrathecal and subcutaneous administration. Further, the pharmaceutical composition may be administered to a subject or to an ex situ perfused organ of subject in hospital via infusion or via injection by a healthcare professional. Compositions for parenteral administration may, for example, be solutions of the EV or nucleic acid molecule or one or more nucleic acid sequences of the disclosure in sterile isotonic aqueous buffer. Where necessary, the parenteral formulations may include for instance solubilizing agents, stabilizing agents and/or a local anaesthetic to ease the pain at the site of the injection.

The EVs of the disclosure are efficient vehicles for delivery of drugs or diagnostic molecules, e.g., if the EVs comprises a biologically compound that is able to bind to a diagnostic target, into the cytosol of target cells. The EVs, nucleic acid molecules and pharmaceutical compositions of the disclosure are, therefore, advantageously used in therapy and diagnosis. Provided is, therefore, an EV, nucleic acid molecule, one or more nucleic acid sequences or pharmaceutical composition according to the disclosure for use as a medicament. Also provided is an EV, nucleic acid molecule, one or more nucleic acid sequences or pharmaceutical composition according to the disclosure for use in therapy. Also provided is an EV, nucleic acid molecule, one or more nucleic acid sequences or pharmaceutical composition according to the disclosure for use as a diagnostic. Also provided an EV, nucleic acid molecule, one or more nucleic acid sequences or pharmaceutical composition according to the disclosure for use in treatment, prevention or diagnosis of disease. Also provided is a method of treatment, prevention or diagnosis of a disease in an individual in need thereof, comprising administering the EV, nucleic acid molecule, one or more nucleic acid sequences or pharmaceutical composition according to the disclosure to the individual.

The disease is preferably selected from the group consisting of a genetic disorder, cancer, a cardiovascular disorder, an infectious disease, an inflammatory disease, a central nervous system disorder, a respiratory disorder, a musculoskeletal disorder, a metabolic disorder and an immunological disorder. Non-limiting examples of diseases that can be treated with the EV, nucleic acid molecule, one or more nucleic acid sequences or pharmaceutical composition according to the disclosure are endocrine disorders, gastrointestinal disorders, renal and urological disorders, haematological disorders, dermatological disorders, ophthalmological disorders, otolaryngologic disorders, obstetrical and gynaecological disorders, psychiatric disorders, neurodegenerative disorders, congenital disorders, autoimmune disorders, allergic disorders, nutritional disorders, substance-related disorders, environmental disorders and rare disorders.

Features may be described herein as part of the same or separate aspects or embodiments of the present disclosure for the purpose of clarity and a concise description. It will be appreciated by the skilled person that the scope of the disclosure may include embodiments having combinations of all or some of the features described herein as part of the same or separate embodiments.

The disclosure will be explained in more detail in the following, non-limiting examples.

### BRIEF DESCRIPTION OF THE DRAWINGS

FIG. 1: Extracellular Vesicles protein delivery through endoprotease-cleavable linkers Schematic illustration of the proposed mechanism of action of the VINCI system for target protein loading inside EVs through a covalent attached endoprotease-cleavable linker. Upon VINCIs internalization in recipient cells followed by endosomal escape leads to the release of the target protein into the cytosol by endoprotease-mediated cleavage.

FIGS. 2A-2F: Characterization of VINCI-GFP EVs (FIG. 2A) Schematic illustration of VINCI1 and VINCI2 constructs with one or two furin-cleavable sites, respectively. (FIG. 2B) NTA analysis showed the size distribution of VINCI1- and VINCI2-GFP EVs to be between ~50-250 nm with a comparable (FIG. 2C) average diameter of 100.0-2.3 and 104=1.8 nm and (FIG. 2D) mode size of 77.0±4.3 nm and 72.1±1.9 nm, respectively. (FIG. 2E) Western blot characterization of VINCI1- and VINCI2-GFP EVs with antibodies against VSV-G, β-actin and common EV-marker proteins TSG101, Synthenin-1 and CD81. (FIG. 2F) Microscopic analysis of transient transfected HEK293FT cells with GFP WT, VINCI1-GFP and VINCI2-GFP plasmid. Scale bar=100 μm. Representative images of three individual experiments.

FIGS. 3A-3D: Furin cleavage of VINCI1 and VINCI2 (FIG. 3A) Predicted sizes of furin-mediated cleavage of the VINCI1- and VINCI2-GFP constructs. (FIG. 3B) Western blot of furin-mediated cleavage of VINCI1-GFP and VINCI2-GFP transiently transfected in HEK293FT cells cultured with or without 50 μM furin inhibitor. (FIG. 3C) Predicted sizes of the furin-mediated cleavage of the VINCI1-Cre and VINCI2-Cre constructs. (FIG. 3D) VINCI1-Cre and VINCI2-Cre transiently transfected in HEK293FT cells cultured with or without 50 μM furin inhibitor.

FIGS. 4A-4F: VINCIs mediate protein loading inside EVs and functional transfer of Cre recombinase in stoplight reporter cells (FIG. 4A) Schematic illustration of the production of VINCI EVs, by transiently transfecting VSV-G with the VINCI1-Cre or VINCI2-Cre plasmid in HEK293FT cells, cultured with or without the furin inhibitor and isolating the EVs via differential ultracentrifugation. (FIG. 4B) Western blot of WT Cre,—VINCI1-Cre and VINCI2-Cre EVs with antibodies against Cre, Alix, CD81, and VSV-G. (FIG. 4C) Quantification of relative Cre protein levels inside EVs. (FIG. 4D) Schematic illustration of Cre-mediated recombination inside Cre-loxP reporter cells (FIG. 4E) Microscopic analysis and (FIG. 4F) Flow cytometry analysis of WT Cre, VINCI1- and VINCI2 mediated Cre delivery led to Cre recombination to eGFP+ reporter cells with an efficiency of 5.8±0.1, 26.1±0.6 and 43.0±0.4% eGFP+ reporter cells. Data expressed at mean±SEM and analyzed using an ordinary one-way ANOVA with *p<0.05 and ** p<0.01.

FIGS. 5A-5H: Sequences of N-myristoylation site (SEQ ID NO:47), linker (SEQ ID NOs: 48, 49, 51, and 53), furin cleavage site(s) (SEQ ID NOs: 1, 14, 25, and 45) and target protein (GFP (SEQ ID NO: 57) or Cre (SEQ ID NOs: 58 and 60)) for the different VINCI plasmids.

FIGS. 6A-6L: Sequences of VINCI1-GFP (SEQ ID NO:57), VINCI1-Cre (SEQ ID NO:58), VINCI2-GFP (SEQ ID NO:59), VINCI2-Cre (SEQ ID NO:60) and VSV-G plasmids.

FIGS. 7A-7F: Variations in membrane anchoring and linker length improve VINCI-mediated intracellular delivery of Cre recombinase. (FIG. 7A) Schematic illustration of membrane anchoring modifications and linker length variations in the VINCI1-Cre plasmid. (FIG. 7B) Western blot analysis of HEK293FT cells transiently transfected with the respective VINCI1-Cre constructs harboring novel membrane anchoring sites or linker lengths. Blots were probed with antibodies against Cre, Furin, VSV-G, β-actin, and EV marker proteins TSG101 and CD9. (FIG. 7C) Western blot analysis of VINCI1-engineered EVs. (FIG. 7D) Quantification of relative Cre protein levels inside EVs harboring novel membrane anchoring sites or linker lengths. Blots were probed with antibodies against Cre, Furin, VSV-G, β-actin, and EV marker proteins TSG101 and CD9. (FIG. 7E) Microscopic analysis and (FIG. 7F) Flow cytometry analysis of VINCI1-Cre EV-mediated Cre delivery to eGFP+ reporter cells. Scale bar=400 μm. Data are expressed as mean±SEM and were analyzed using an ordinary one-way ANOVA with *p<0.05, ** p<0.01, *** p<0.001, and **** p<0.0001. Data are from two independent experiments.

FIGS. 8A-8F: Furin protease cleavage site within the VINCI-1 construct enables efficient intracellular delivery of Cre recombinase. (FIG. 8A) Schematic illustration of the new protease cleavage sites (CS) introduced in the VINCI1-Cre plasmid including sites for trypsin, ADAM17 (A Disintegrin And Metalloproteinase 17), Matrix Metalloproteinase (MMP) CS 1, and MMP CS 2. (FIG. 8B) Western blot analysis of HEK293FT cells transiently transfected with the respective VINCI1-Cre constructs harboring novel protease cleavage sites. Blots were probed with antibodies against Cre, Furin, VSV-G, β-actin, and EV marker proteins TSG101 and CD9. (FIG. 8C) Western blot analysis of VINCI1-engineered EVs harboring new protease cleavage sites. Blots were probed with antibodies against Cre, VSV-G, and EV marker proteins TSG101 and CD9. (FIG. 8D) Quantification of relative Cre protein levels inside EVs. (FIG. 8E) Microscopic analysis and (FIG. 8F) Flow cytometry analysis of VINCI1-Cre mediated Cre delivery to eGFP+ reporter cells. Scale bar=400 μm. Data are expressed as mean±SEM and were analyzed using an ordinary one-way ANOVA with *p<0.05, ** p<0.01, *** p<0.001, and **** p<0.0001. Data are from a single experiment.

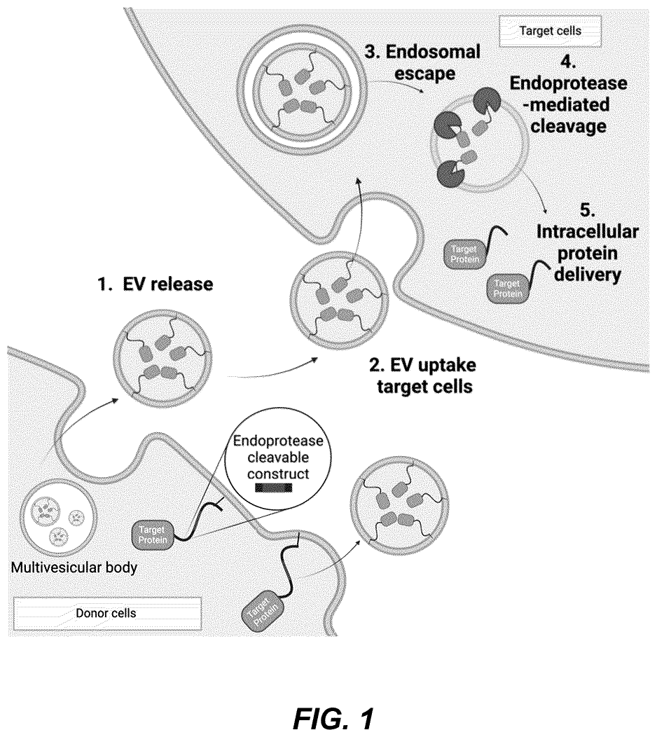

### DETAILED DESCRIPTION

### Examples

### Materials and Methods

### Cell Culture

Human embryonic kidney 293 FT (HEK293FT) (R70007, ThermoFisher) and T47D stoplight reporter cells provided by L. Jiang (Zomer et al. 2016) were maintained in Dulbecco's Modified Eagle Medium (DMEM) supplemented with 10% fetal bovine serum (FBS), 1% penicillin/streptomycin (P/S). All cells were maintained in a 5% CO2 environment at 37 °C.

### Plasmids

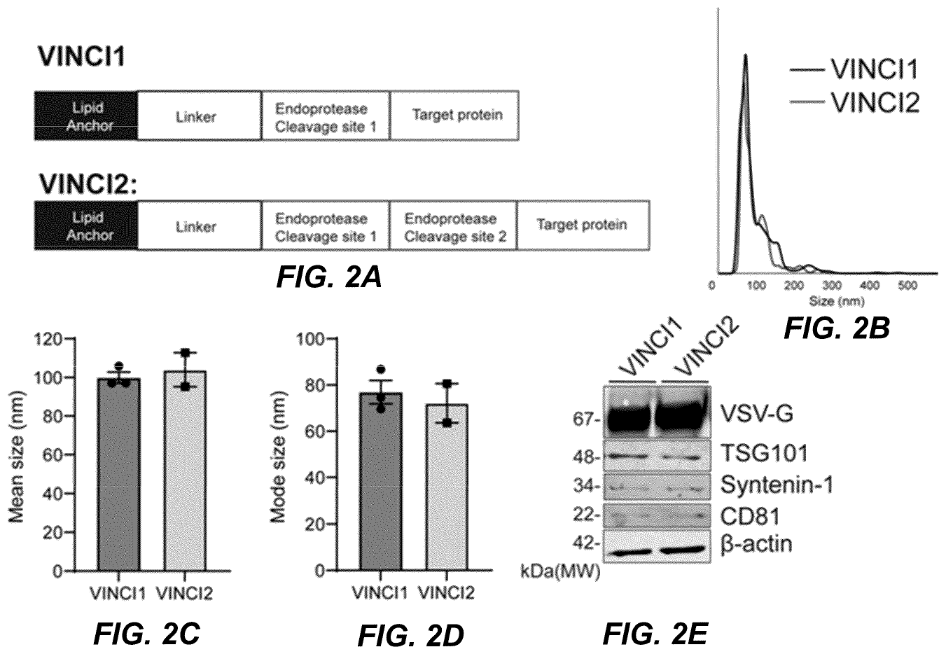

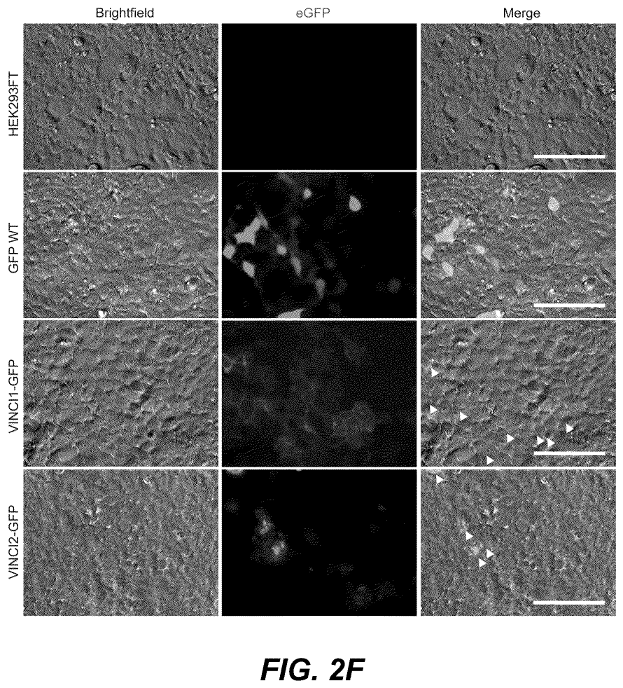

The VINCI1-Cre and VINCI1-GFP plasmids were constructed by first performing a PCR amplification of Cre or GFP sequence. Subsequently, the respective sequences (lipid anchor sequence, linker, endoprotease cleavage site and target protein; see FIG. 2A) were cloned into a pcDNA5/FRT/KO plasmid (V6520-20, ThermoFisher) with the NEBuilder® HiFi DNA Assembly kit (E2621L, BIOKÉ) according to the manufacturer's protocol.

To create the VINCI2-Cre and VINCI2-GFP plasmid, the oligos of the second furin cleavage site (see FIG. 2A) was annealed and inserted into digested VINCI2-Cre and VINCI2-GFP plasmids with T4 ligase (M0202S, NEB) by following the manufacturer's instructions.

The VSV-G plasmid (pCMV-VSV-G; Addgene Plasmid #8454) used was as described in Stewart et al. (Lentivirus-delivered stable gene silencing by RNAi in primary cells; RNA 2003 April;9 (4): 493-501. 10.1261/rna.2192803 PubMed 12649500).

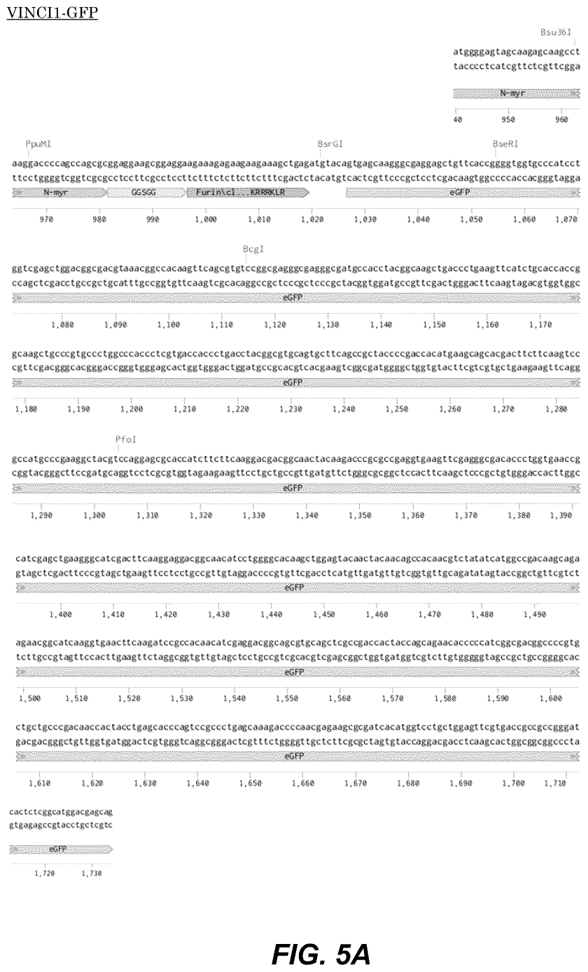

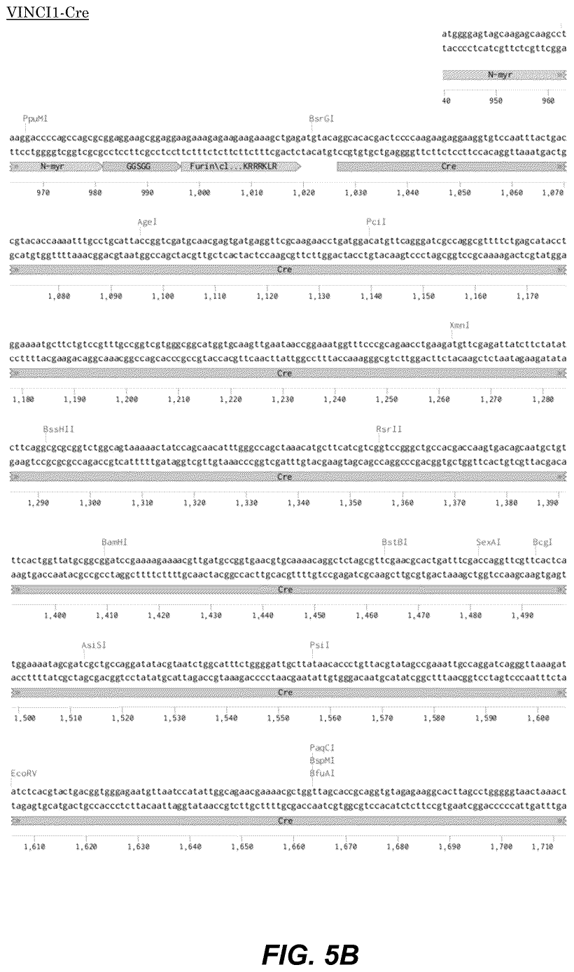

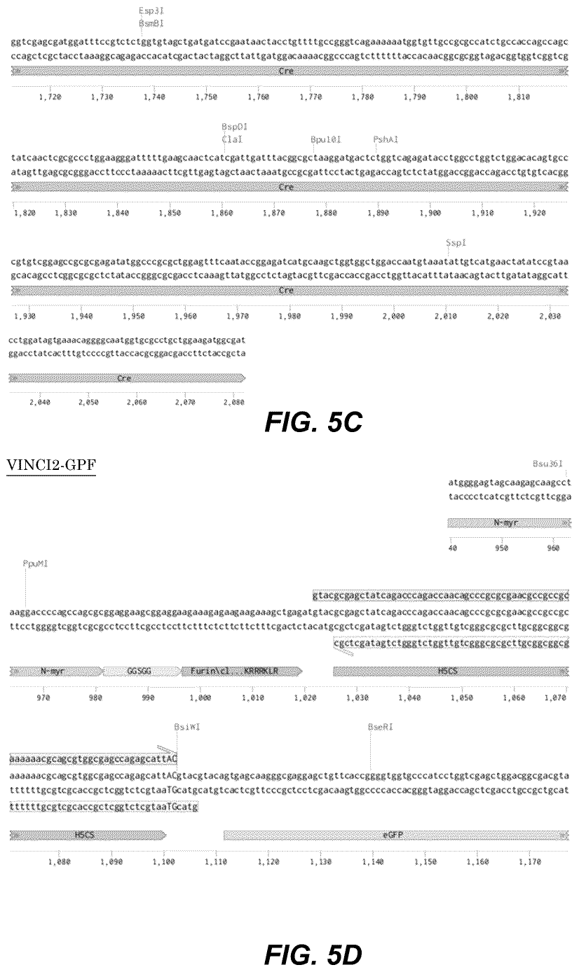

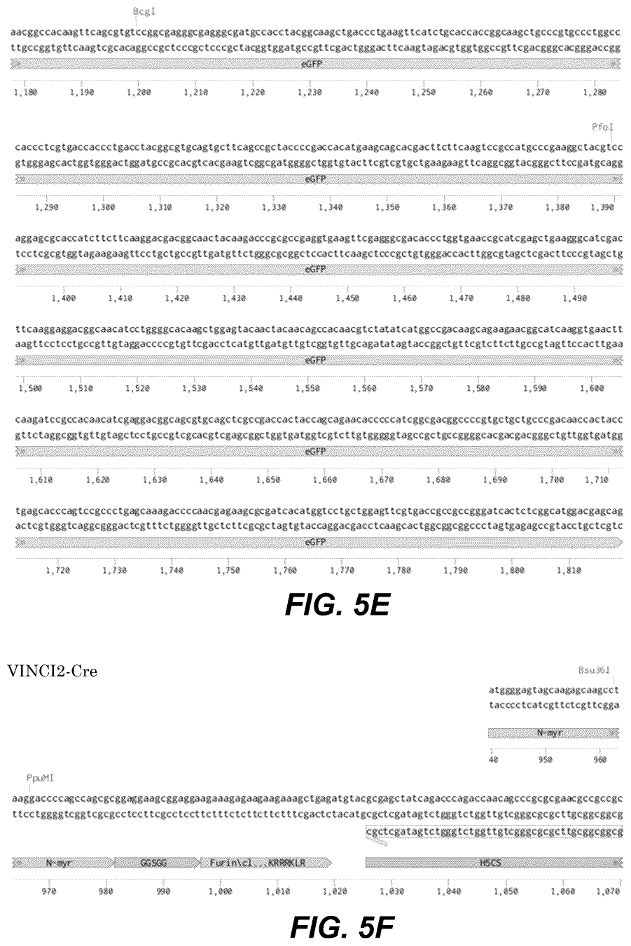

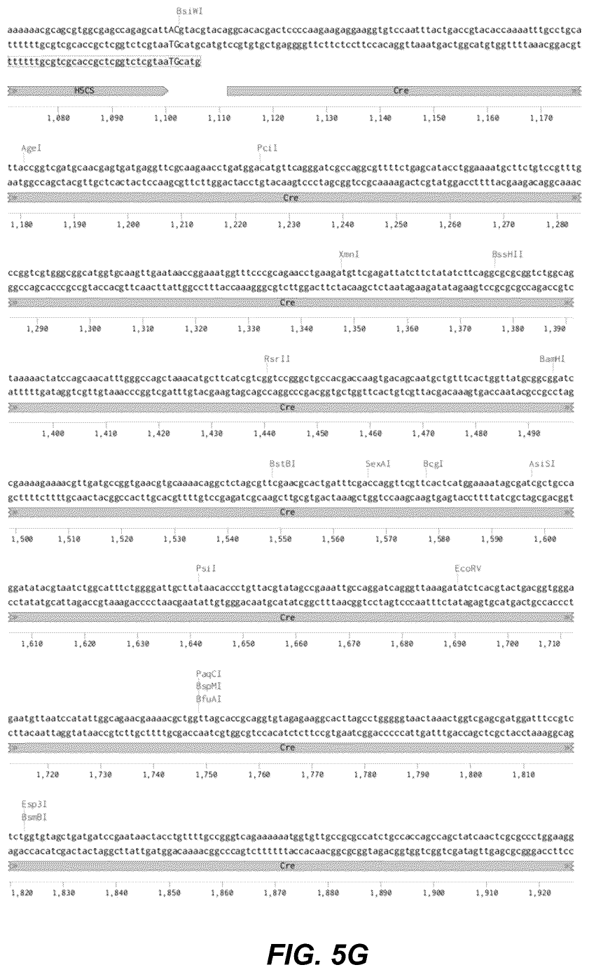

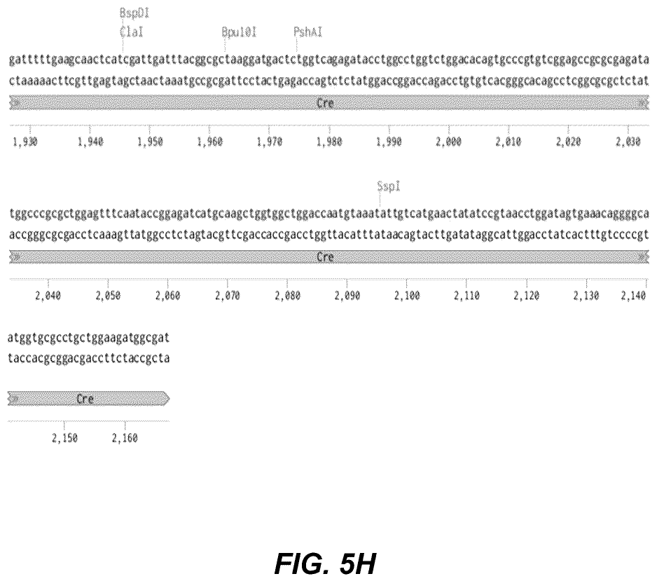

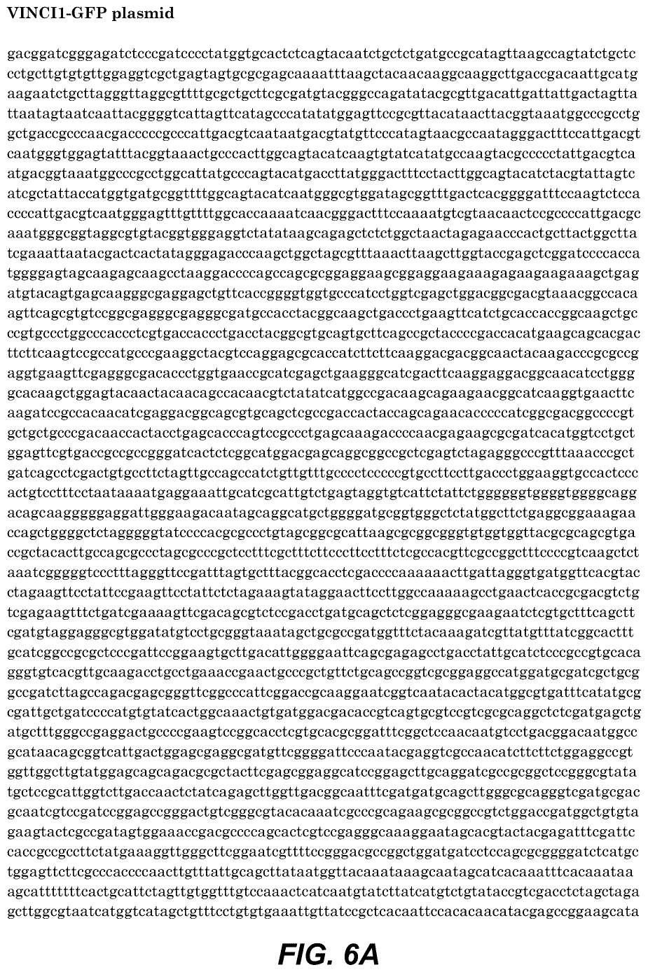

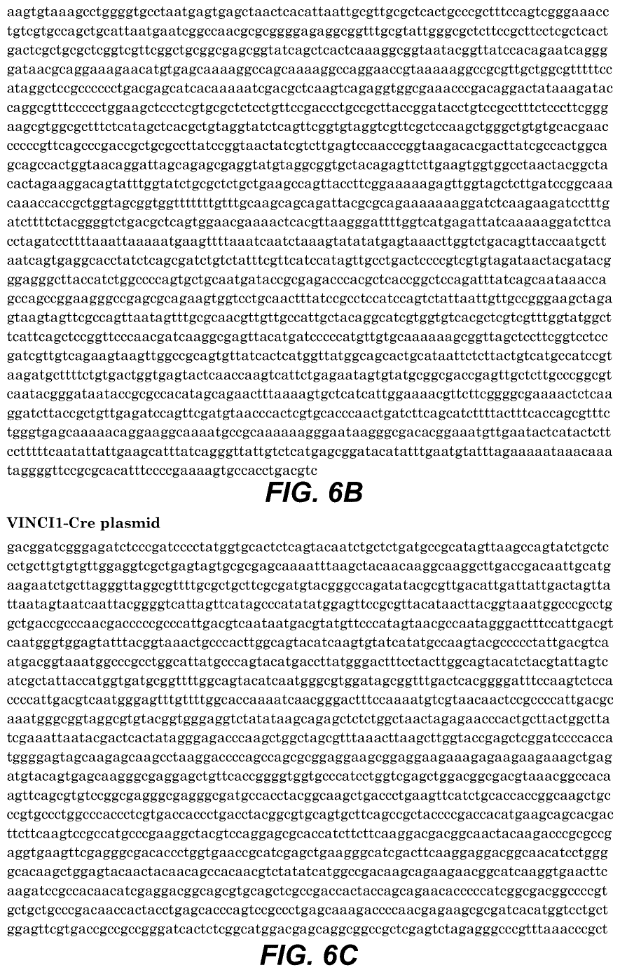

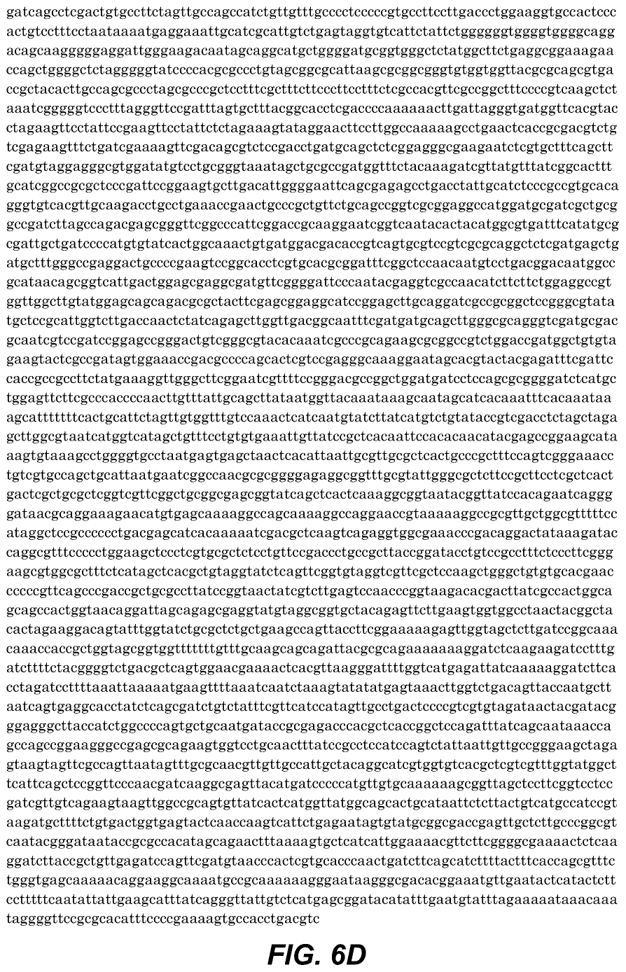

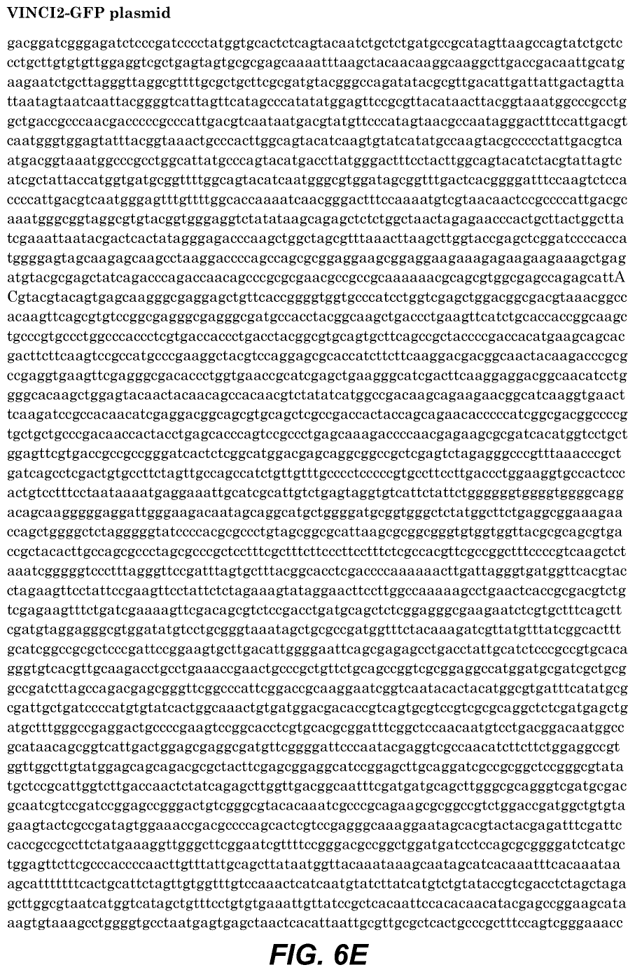

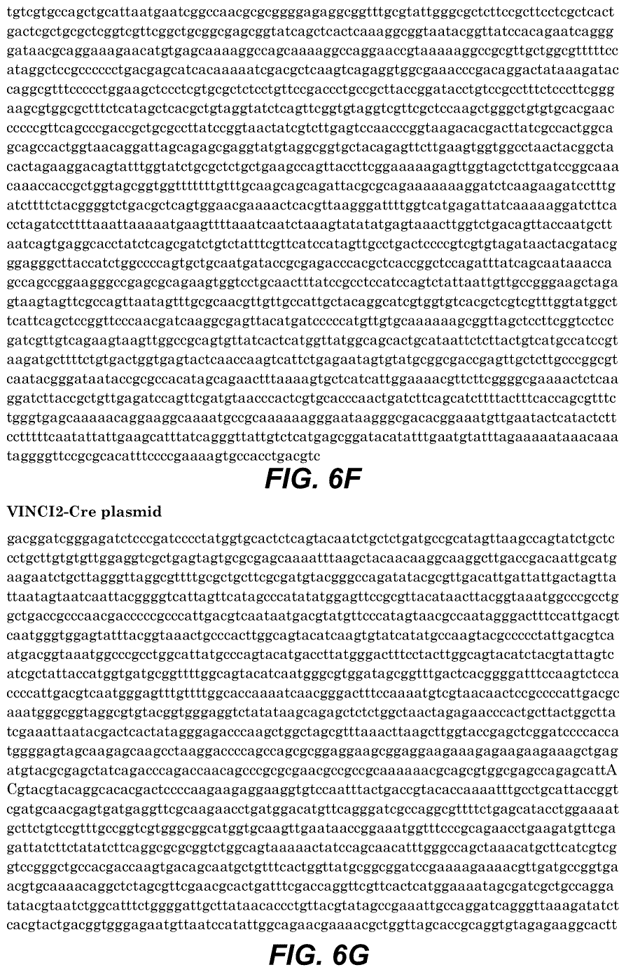

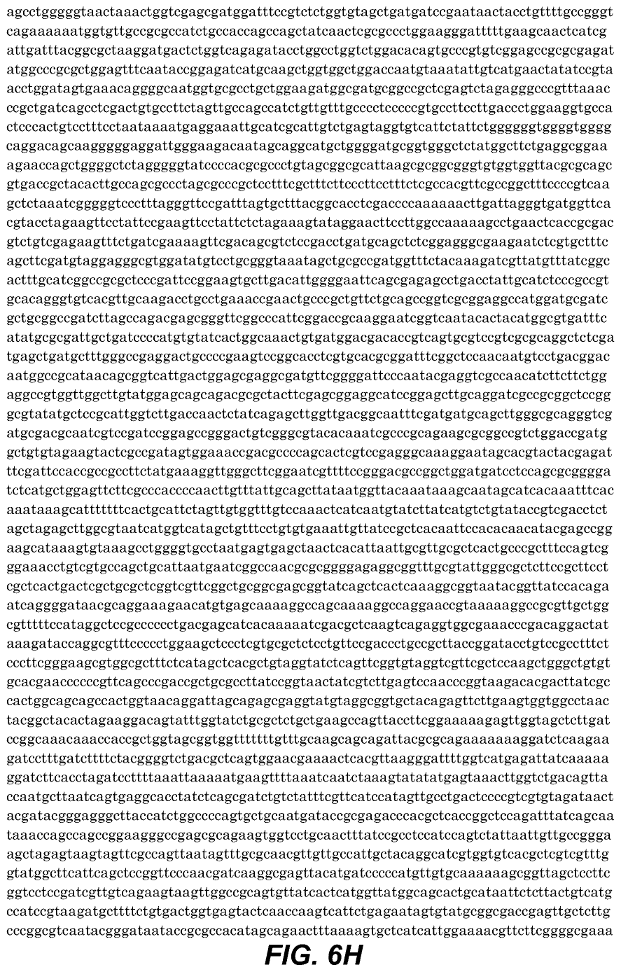

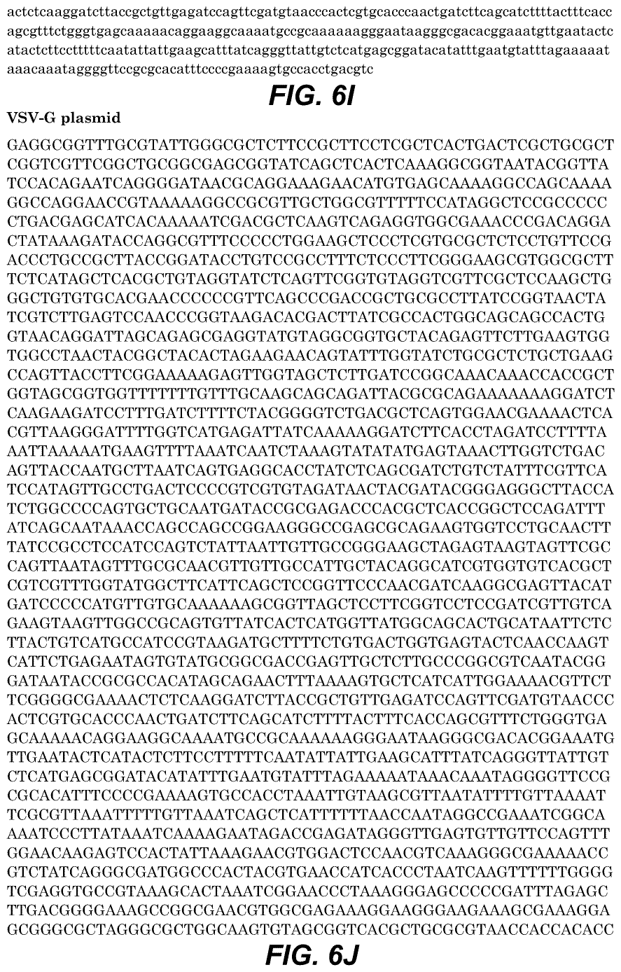

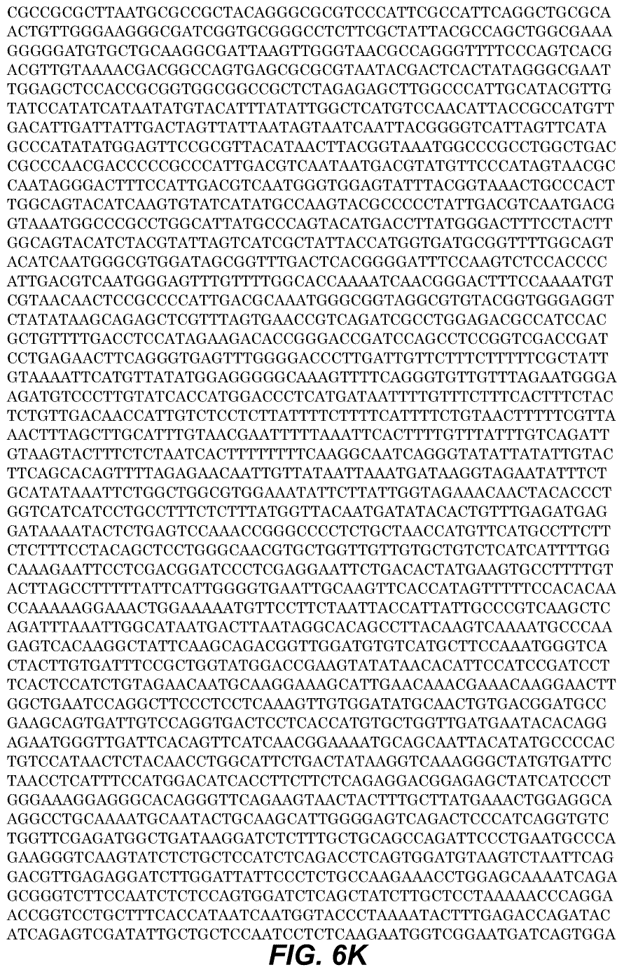

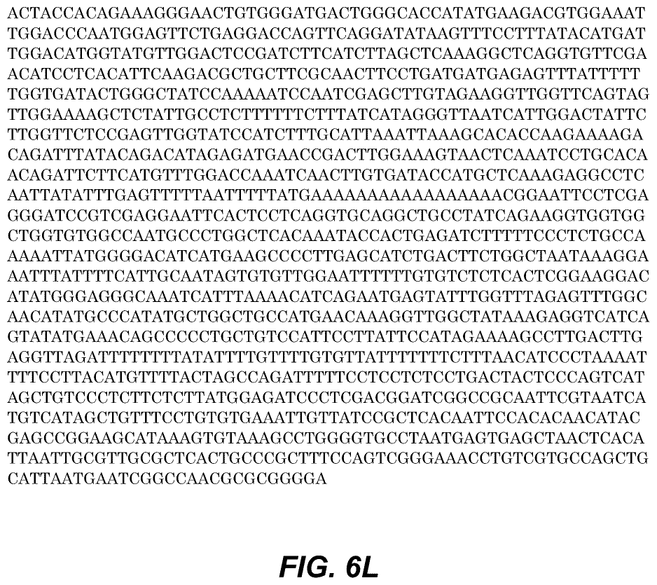

Sequences of N-myristoylation site, linker, furin cleavage site and target protein for the different VINCI plasmids are shown in FIGS. 5A-5H. Full sequences of the VINCI and VSV-G plasmids are depicted in FIGS. 6A-6L.

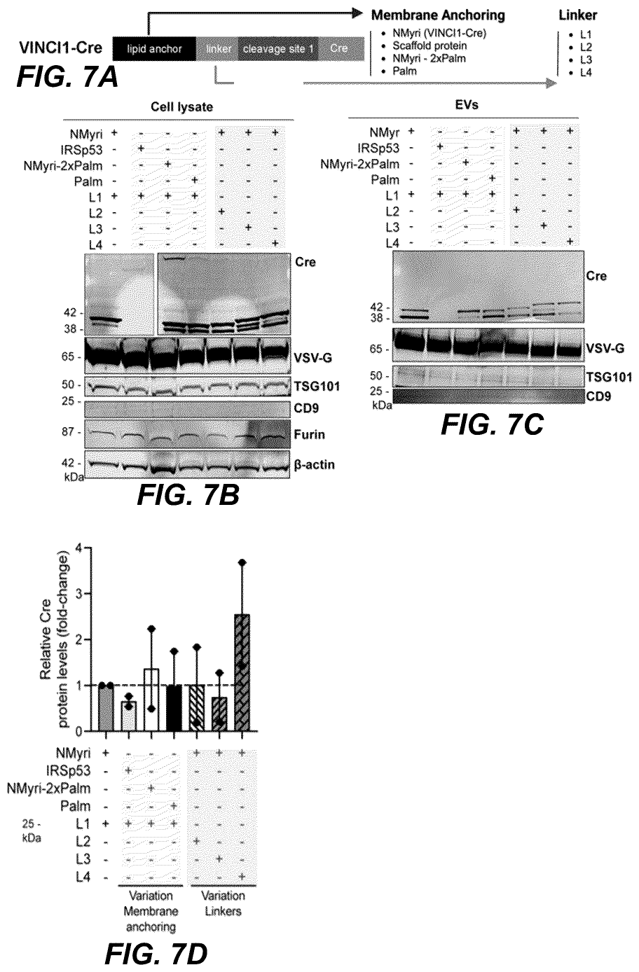

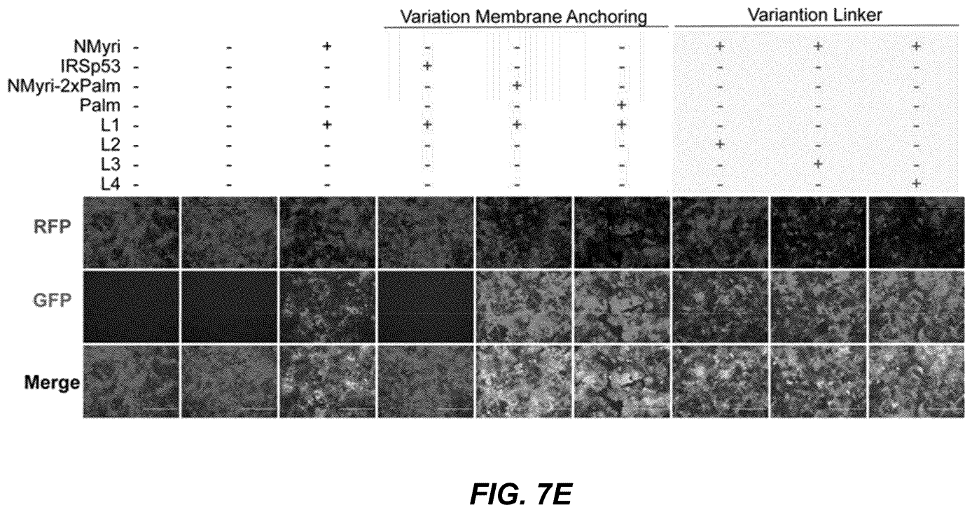

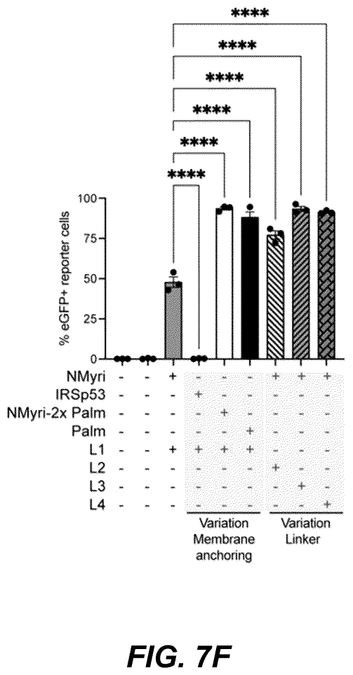

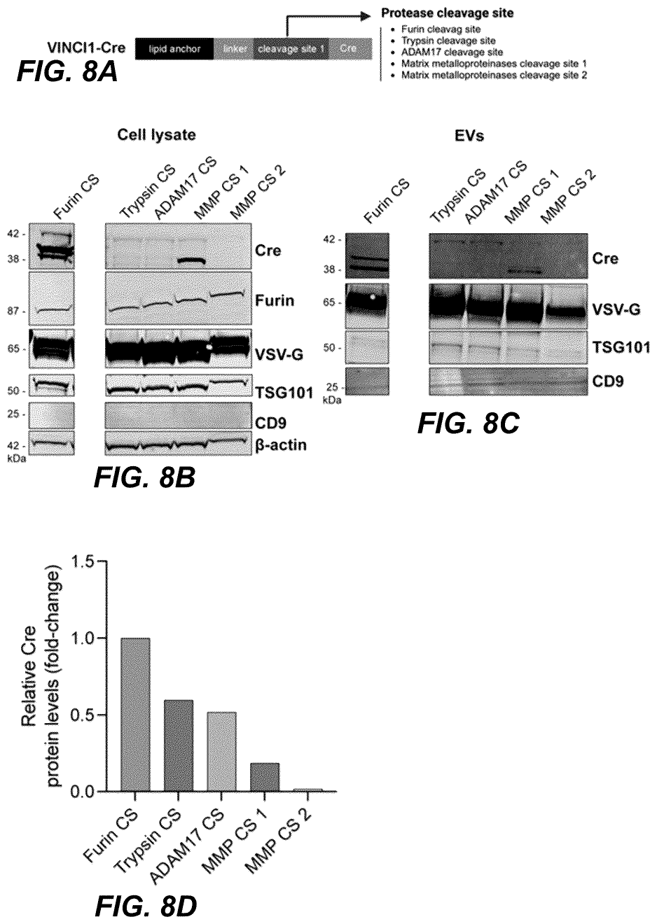

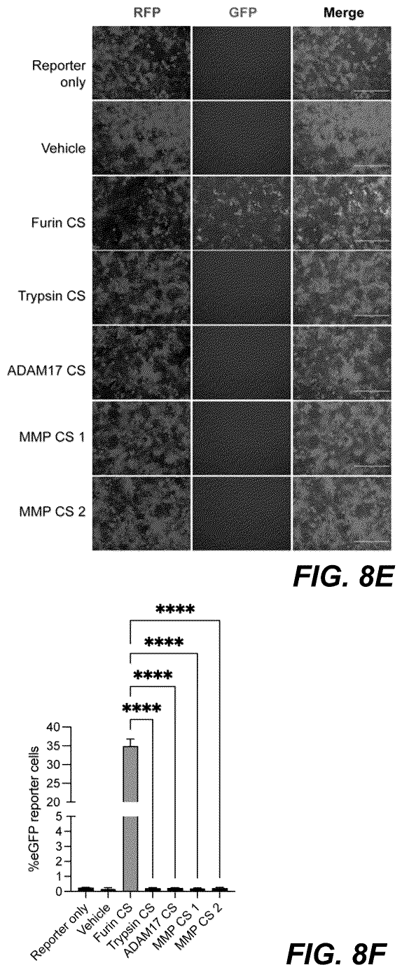

Additional plasmids were prepared in which the membrane anchor sequence, protease cleavage site and linker sequence between membrane anchor and cleavage site was varied. As the membrane anchor a palmitoylation anchor, a myristoylation-palmitoylation-palmitoylation lipid anchor (N-Myri-2×Palm) and membrane protein anchor (IRSp53) were introduced in addition to an N-myristoylation anchor (see FIG. 7A); four different linking sequences (L1: GSGGTG (SEQ ID NO:67); L2: GSGGGSGGGGTG (SEQ ID NO:68); L3: GSKESGSVSSEQLAQFRSLDTG (SEQ ID NO:69); L4: GSKEAAAKDR (SEQ ID NO:73)) were introduced (see FIG. 7A). Finally, the following endoprotease cleavage sites were introduced in addition to furin (see FIG. 8A): trypsin (active in the extracellular space), ADAM17 (active extracellularly) and two matrix metalloproteinase (MMP) cleavages sites (active in extracellular space).

### EV Production and Isolation

In a T175 flask, 1×10⁷ HEK293FT cells were seeded and cultured overnight. The next day, the medium was changed to DMEM supplemented with 10% exosome-depleted FBS (A2720801, Gibco). Subsequently, the cells were transfected with either 30 μg of VINCI1-GFP, VINCI2-GFP, GFP WT, VINCI1-Cre, VINCI2-Cre, WT Cre together with 15 μg VSV-G plasmid by mixing the plasmid and Lipofectamine® 3000 Transfection Reagent (L3000008, Invitrogen Corp.) in a 1:2 ratio and following the manufacturer's instructions. After 6 hours, the medium was changed to DMEM supplemented with 10% FBS and 1% P/S. After 48 hours, the medium was changed to Opti-MEM (31985-070, Gibco) supplemented with or without the furin inhibitor (R&D Systems). After 24 hours, the conditional medium was recovered and centrifugated at 2000× g for 15 minutes at 4 °C to remove cell debris. Next, the supernatant was centrifuged at 10,000×g for 30 minutes at 4 °C using an Open-Top Thinwall Polypropylene Tube, 25×89 mm 50 Pk (326823, Beckman Coulter) and SW 32 Ti Swinging-Bucket (369694, Beckman Coulter). Next, the supernatant was recovered and centrifuged at 100,000×g for 70 minutes at 4 °C to pellet the EVs. The EV pellet was recovered by suspending the pellet in PBS and filtered through a 0.45 μm SCFA membrane syringe filter (516-1954, Corning).

### Nanoparticle Tracking Analysis

The size and particle concentration of EVs were assessed with nanoparticle tracking analysis (NTA) (NS500, Malvern Nanosight). EVs were suspended in PBS and measured in triplicate with individual measurements of 30 seconds at camera level 15. Analysis was performed with NTA software 3.3 with a minimal track length of 10, detection threshold 5, and screen gain 1. The cut-off values for reliable measurement were between 20.0 and 100.0 particles/frame.

### Western Blot

Cell lysates and EVs were lysed in RIPA lysis buffer (1:10, 20-188, Sigma) supplemented with Protease/Phosphatase inhibitor cocktail (1:100, 5872S, Cell Signaling Technology) and stored on ice for 30 minutes-2 hours. Next, only the GFP WT, WT Cre, N-Myr-FBKP12/FRB-Cre samples were centrifuged at 14,000× g for 10 minutes at 4 °C where the supernatant was isolated. The lysed EVs and cell lysates were stored at −20 °C.

Protein concentrations were determined via the Micro BCA Protein Assay Kit (23235, ThermoFisher). Samples were mixed with NuPAGE™ Sample Reducing Agent (50 mM, NP0004, Invitrogen Corp), LDS sample buffer (Life Technologies), 5% (v/v) 2-betamercap ethanol, and heated at 95 °C for 10 minutes. Samples were separated on Bolt™ 4-12% Bis-Tris Plus Gel (NW04125BOX, ThermoFisher Scientific). With a PageRuler Plus Prestained Protein Ladder (26619, ThermoFisher Scientific) at 130 V for 75 minutes and transferred to PVDF membranes (IPVH00010, Merck). The membranes were blocked for 1 hour in 5% bovine serum albumin (BSA) (w/v) in Tris-buffered saline (TBS).

Primary antibodies included mouse anti-Alix (1 μg/mL, abcam, ab41927), mouse anti-syntenin (1 μg/mL, TA504796, Origene), mouse anti-CD81 (1 μg/mL, SC-166029, Santa Cruz), anti-rabbit GFP (1 μg/mL, Cell Signaling Technology, #2956, clone D5.1), anti-rabbit Cre recombinase (1 μg/mL, Cell Signaling Technology, #15036, clone D7L7L), anti-rabbit furin antibody (1 μg/mL, Abcam) and mouse anti-β-actin (0.2 μg/mL, Cell Signaling Technology, clone 8H10D10). Secondary antibodies included Alexa Fluor 680-conjugated anti-mouse antibody (0.1 μg/mL, LI-COR Biosciences, A-21057) and IRDye 800CW anti-rabbit antibody (0.1 μg/mL, 1926-322, LI-COR Biosciences). Imaging was performed on an Odyssey Infrared Imager (LI-COR Biosciences) at 700 nm and 800 nm.

### Cre Reporter Assay

In a flat bottom 96-well plate (655075, Greiner CELLSTAR), T47D stoplight reporter cells were seeded at a density of 10,000 cells/well and incubated o/n. The next day, the conditional media or differential ultra-centrifugated EVs were added to T47D stoplight reporter cells. After 6 days, the cells were directly visualized with EVOS Cell Imaging System (M5000, Invitrogen) and subsequently analyzed by flow cytometry (CytoFLEX, Beckman Coulter).

### Flow Cytometry Analysis

Cells were washed with PBS and dissociated with 0.25% Trypsin-EDTA solution (Sigma-Aldrich, T4049). The dissociated cells were transferred to a 96-well round bottom plate (650185, Greiner CELLSTAR®), centrifugated at 500×g for 3 minutes, and resuspended in 250 μl PBS supplemented with 2% FBS. Fluorescence data were acquired by employing a CytoFLEX flow cytometer (Beckman Coulter, Inc.) and analyzing using Kaluza software v2.1 (Beckman Coulter, Inc.).

### Microscopy Analysis

Microscopy analysis was performed with the EVOS FL Cell Imaging System (LifeTechnologies).

### Statistical Test

The statistical analysis was carried out using GraphPad PRISM v.9.3. Comparisons between two groups were analyzed using a two-tailed unpaired t-test as indicated. Comparisons between multiple groups were analyzed by one-way ANOVA with Tukey's multiple comparison post-test. Data is represented as mean±SEM with p-values *<0.05 and **<0.01 were considered statistically significant.

### Results

### Characterization of VINCIs

The VINCI system was created by fusing the covalent lipid anchor linker to a single furin cleavage site (VINCI1; KRRKLR (SEQ ID NO:1)) or two furin cleavages sites (VINCI2; KRRKLR (SEQ ID NO:1) and ASYQTQTNSPRERRRKKRSVASQSI (SEQ ID NO: 47)) (FIG. 2A). Subsequently, the GFP or Cre recombinase sequence (target protein) was fused to the C-terminal of the furin cleavage site(s).

The VINCI-GFP EVs were collected and isolated from transiently transfected HEK293FT cells with the VINCI1-GFP or VINCI2-GFP plasmid and the VSV-G plasmid, and isolated by differential ultracentrifugation. The mean EV size was assessed for VINCI1- and VINCI2-GFP EVs using nanoparticle tracking analysis (NTA) and found to be 100.0=2.3 and 104±1.8 nm, respectively (FIGS. 2B, 2C). The mode size determined by NTA for VINCI1- and VINCI2-GFP EVs were 77.0±4.3 nm and 72.1±1.9 nm, respectively (FIG. 2D). Western blot analysis showed VINCI1- and VINCI2-GFP EVs contained VSV-G, β-actin, Synthenin-1, CD81 and TSG101 (FIG. 2E). These findings showed that VINCI EVs were successfully isolated from transiently transfected HEK293FT cells.

To determine the intracellular location of the GFP inside donor cells, HEK293FT cells were transiently transfected with the WT GFP, VINCI1-GFP, or VINCI2-GFP plasmid. Microscopic fluorescent analysis showed WT GFP is located within the cytosol while VINCI1-GFP and VINCI2-GFP are linked to the (sub) cellular membrane via a covalent lipid modification (FIG. 2F). These results demonstrated that GFP, from the VINCI1 and VINCI2 constructs, is successfully anchored to the cellular membrane through the lipid modification of an N-Myristoylation sequence.

### Cleavage of VINCI1 and VINCI2

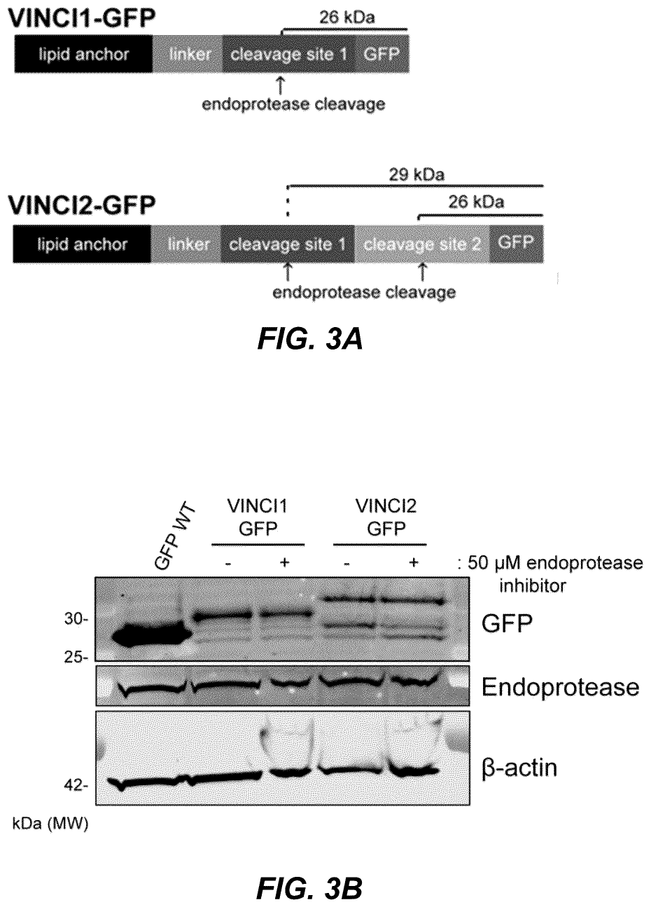

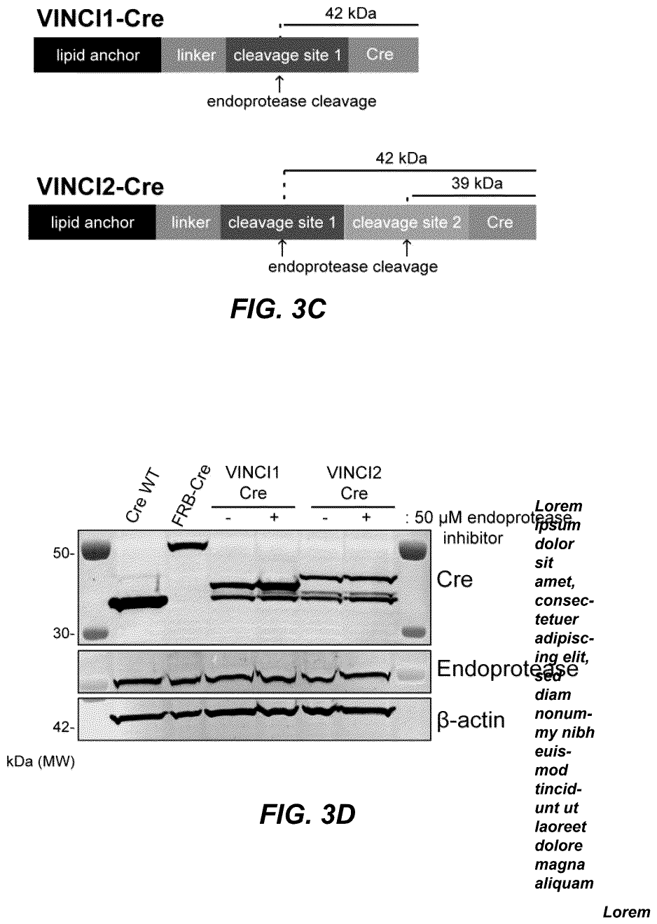

To validate the furin-mediated cleavage within the linker regions, VINCI1- and VINCI2-GFP plasmids were transiently transfected in HEK293FT cells. A small molecule furin inhibitor was co-incubated with the transiently transfected cells to investigate if furin-mediated cleavage could be blocked. Furin cleavage of VINCI1-GFP and VINCI2-GFP yielded one GFP cleavage (26 kDa) and two GFP products (26 and 29 kDa) (FIG. 3A). Western blot analysis showed that VINCI1-GFP (26 kDa) and VINCI2-GFP (36 kDa) were cleaved at the predicted sizes by the presence of one GFP (26 kDa) or two GFP ( 26/29 kDa) bands (FIG. 3B), respectively. Since a reduction in GFP cleavage products with co-incubation of the furin inhibitor was not observed, it suggests that the concentration was insufficient to inhibit furin-mediated cleavage.

To study whether furin-mediated cleavage with the VINCI-Cre constructs could be inhibited, the VINCI1-Cre and VINCI2-Cre plasmids were transiently transfected in HEK293FT cells and cultured with or without 50 μM furin inhibitor. As a negative control, Cre was loaded inside EVs through a rapamycin induced FKBP12/FRB dimerization system. Similarly to the VINCI-GFP constructs, Western blot analysis showed furin-mediated cleavage of VINCI1-Cre and VINCI2-Cre resulted in one (39 kDa) or two cleave products ( 39/42 kDa) of Cre recombinase, respectively (FIGS. 3C, 3D). Co-incubation with 50 M of the furin inhibitor only slightly reduced the furin-cleavage of the VINCI1-Cre construct. Observations from this experiment suggest that co-culture with 50 μM of CMK of the furin inhibitor did not reduce the furin in all constructs.

### Enhanced Loading of Cre Recombinase Inside VINCI EVs

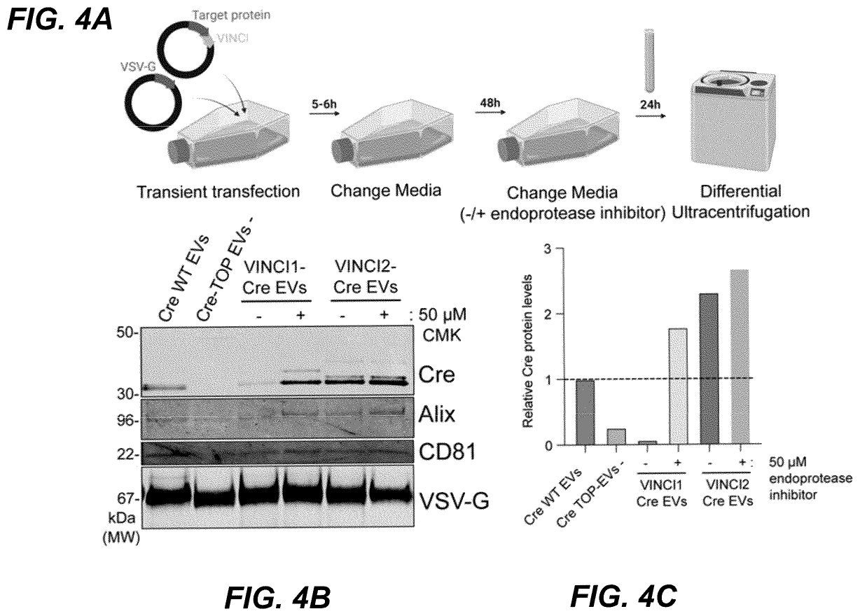

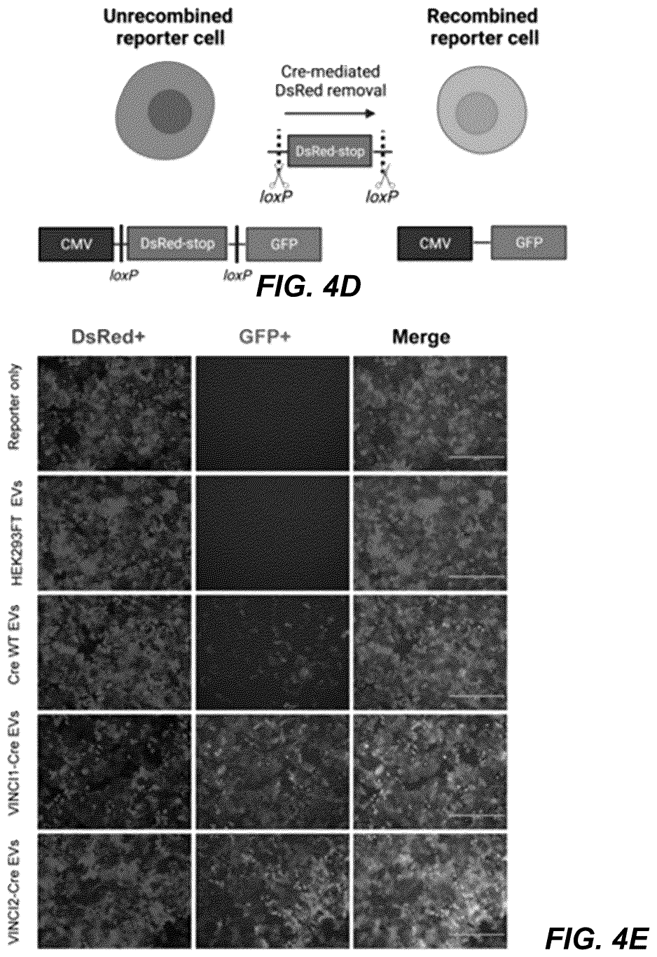

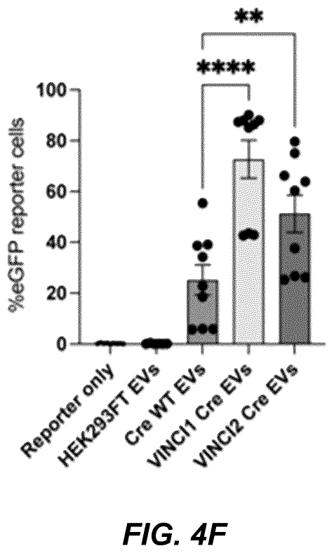

To produce VINCI EVs, it was hypothesized that the expression of VINCI1 or VINCI2 linked to a target protein in endoprotease knockout donor cells would further promote protein loading within EVs. However, an endoprotease knockout donor cell was not found. Therefore, VINCI-mediated protein loading in EVs was investigated by co-culturing the donor cells in the presence of the endoprotease inhibitor. HEK293FT cells were transiently transfected with VSV-G, and the VINCI1- or VINCI2 plasmid fused to Cre recombinase. After 48 hours, the medium was changed to serum-free medium supplemented with or without the small molecule endoprotease inhibitor. After 24 hours, the conditional medium was collected and the EVs were obtained by differential ultracentrifugation (FIG. 4A).

VINCI1 and VINCI2-mediated loading of Cre recombinase inside EVs was analyzed by comparing the EV protein content derived from transiently transfected VINCI1 or VINCI2 donor cells cultured in the presence or absence of the endoprotease inhibitor. EVs derived from donor cells transiently transfected with Cre-FRB/2×FKBP12 plasmid and cultured without rapamycin orthologue were included. The extent of Cre recombinase loading within EVs was normalized by WT Cre loaded in EVs through overexpression in donor cells.

Western blot analysis showed that Cre recombinase loading within EVs through the VINCI1 construct was increased by 1.7-fold compared to the WT Cre loaded inside EVs. As observed in the cell lysate of VINCI1-Cre transfected donor cells (FIG. 3D), co-incubation with the endoprotease inhibitor slightly reduced endoprotease-mediated cleavage of Cre recombinase, indicating that it also promoted Cre recombinase loading via VINCI1 within EVs. These observations suggest that the VINCI1 construct can only load Cre recombinase inside EVs when co-cultured with an endoprotease inhibitor.

The VINCI2 construct promoted Cre recombinase loading by 2.1- and 2.4-fold when the endoprotease inhibitor was absent or present, respectively. Co-culturing with the endoprotease inhibitor only resulted in a small increase of Cre recombinase loading inside EVs. As observed in the cell lysate of transiently transfected VINCI2-Cre donor cells (FIG. 3B), co-culturing with the endoprotease inhibitor still resulted in two endoprotease cleaved products. These observations suggest that co-culturing with the endoprotease inhibitor does not affect VINCI2 ability to load Cre recombinase within EVs.

### Functional Transfer of Cre Recombinase Through VINCI-EV

To examine if VINCI-EVs could transfer functional proteins to target cells, VINCIs-mediated transfer of Cre recombinase to T47D stoplight reporter system Klik of tik om tekst in the voeren. were tested. Upon successful intracellular delivery of Cre recombinase-mediated by EV within Cre-loxP reporter cells, Cre recombination of the loxP sites results in a color switch from DsRed+ to eGFP+ reporter cells (FIG. 4D). VINCI1- and VINCI2-Cre EVs were obtained by transfection of HEK293FT cells with VSV-G together with WT Cre, VINCI1-Cre or VINCI2-Cre plasmid. After 72 hours post-transfection, the EVs were obtained via differential ultracentrifugation, and 1×1011 number of particles of WT Cre EVs, VINCI1- or VINCI2-Cre EVs were administrated to T47D stoplight reporter cells. After six days post administration, microscopic- and flow cytometry analysis showed that WT, VINCI1- and VINCI2 EVs delivered Cre recombinase, leading to 5.8±0.1%, 26.1±0.6% and 43.0±0.4% eGFP+ recombined reporter cells, respectively (FIGS. 4E, 4F). VINCI engineered EVs enable more recombination in target cells more effectively than WT Cre EVs. In line with the Western blot findings (FIGS. 4B, 4C), it was shown that the increased Cre loading inside EVs through the VINCI2 construct resulted in significantly higher Cre recombinase transfer by VINCI2-Cre EVs compared to VINCIs1-Cre EVs. Taken together, VINCI1 and VINCI2 EVs enabled the functional delivery of Cre recombinase in T47D stoplight reporter cells.

### Varying Membrane Anchor Sequence and Linker Sequence

Variations in membrane anchoring and linker length improve VINCI-mediated intracellular delivery of Cre recombinase. Membrane anchoring was substituted from the standard N-Myristoylation tag (NMyri) to include a scaffold protein (IRSp53), N-Myristoylation-Palmitoylation-Palmitoylation (NMyri-2×Palm), or a single palmitoylation (Palm) sequence. Additionally, linker lengths were modified to include a flexible linker (short; L2), flexible linker (long; L3), or a rigid linker (L4) (FIGS. 7A-7F). The analysis showed Cre recombination efficiencies in eGFP+ reporter cells with different membrane anchoring sites as follows: NMyri (original VINCI1-Cre plasmid, 47.8±5.6%), transmembrane protein (0.3±0.2%), NMyri-2×Palm (93.6±1.6%), and Palm (88.2±5.5%). Cre recombination efficiencies in eGFP+ reporter cells with different linker lengths were as follows: L2 (77.7±4.5%), L3 (93.4±2.6%), and L4 (91.3±1.0%).

### Varying Endoprotease Cleavage Site

Furin protease cleavage site within the VINCI-1 construct enables efficient intracellular delivery of Cre recombinase, whereas extracellularly active endoproteases do not (FIGS. 8A-8F). The analysis showed Cre recombination efficiencies in eGFP+ reporter cells for the respective protease cleavage sites as follows: furin (34.9±3.3%), trypsin (0.2±0.1%), ADAM17 (0.2±0.1%), MMP CS 1 (0.2±0.1%), and MMP CS 2 (0.2±0.1%).

### REFERENCES

Corso, G. et al. Systematic characterization of extracellular vesicle sorting domains and quantification at the single molecule-single vesicle level by fluorescence correlation spectroscopy and single particle imaging. J. Extracell. Vesicles 8, 1663043 (2019).
Heusermann, W. et al. Exosomes surf on filopodia to enter cells at endocytic hot spots, traffic within endosomes, and are targeted to the ER. Journal of Cell Biology 213, 173-184 (2016).
Whitley, J. A. et al. Encapsulating Cas9 into extracellular vesicles by protein myristoylation. J. Extracell. Vesicles 11, e12196 (2022).
Zomer, A., Steenbeek, S. C., Maynard, C. & van Rheenen, J. Studying extracellular vesicle transfer by a Cre-loxP method. Nat. Protocols 11, 87-101 (2016).

1. An extracellular vesicle (EV) comprising a biologically active compound covalently attached to the internal surface of the EV membrane via at least one endoprotease-cleavable linker.

2. The EV according to claim 1, wherein the endoprotease is an intracellular endoprotease.

3. The EV according to claim 1, wherein the biologically active compound is covalently attached to the EV membrane via an EV membrane lipid.

4. The EV according to claim 1, wherein the biologically active compound is covalently attached to the EV membrane by S-acylation, palmitoylation, prenylation, N-myristoylation, and/or or a GPI anchor.

5. The EV according to claim 1, wherein the biologically active compound is covalently attached to the EV membrane by N-myristoylation and palmitoylation.

6. The EV according to claim 1, wherein the endoprotease is selected from the group consisting of serine proteases, such as furin, tissue plasminogen activator, activated protein C, coagulation factors, factor VIIa, IXa, Xa, XIa, XIIa, thrombin, plasmin, granzymes, cathepsins, urokinase, matrix metallopeptidases, metalloprotease-related proteins, kallikreins, kallikrein related peptidases, plasma kallikrein, aminopeptidases, chymopasin, prosemin, trypsin, tryptases, acrosin, hepsin, neurotrypsin, ADAMTS peptidases, ADAM peptidases, calpains, dipeptidyl-peptidases, pepsin, gastricsin, presenilin, signal peptide peptidase-like protein, calpamodulin, ubiquitinyl hydrolases, ubiquitin-specific peptidases, SENP peptidases, carboxypeptidases, metallocarboxypeptidases, dihydropyrimidinase related proteins, tryptase beta, complement components, matriptase, gamma-glutamyltransferase, gamma glutamyltransferase-like proteins, separase, proprotein convertases, caspases, and combinations thereof.

7. The EV according to claim 1, wherein the endoprotease is selected from the group consisting of furin, calpain, and combinations thereof.

8. The EV according to claim 1, wherein the biologically active compound is covalently attached to the EV membrane by palmitoylation, prenylation and/or N-myristoylation and wherein the at least one endoprotease-cleavable linker is at least one furin-cleavable linker, and/or at least one calpain-cleavable linker.

9. The EV according to claim 1, wherein the EV expresses Vesicular Stomatitis Virus G (VSV G) protein or a homolog thereof, syncytin-1 and syncytin-2, herpes simplex virus glycoprotein B (HSV gB), rabies viral glycoprotein, influenza virus hemagglutinin (HA), human immunodeficiency virus envelope glycoprotein gp160 (gp160), Caenorhabditis elegans Eff-1, and/or Caenorhabditis elegans Aff-1 on the external surface of the EV membrane.

10. The EV according to claim 1, wherein the biologically active compound is selected from the group consisting of proteins, polypeptides, peptides, small molecules, oligonucleotides, and combinations thereof.

11. The EV according to claim 1, wherein the biologically active compound is selected from the group consisting of proteins, polypeptides, and peptides.

12. The EV according to claim 1, wherein the biologically active compound is selected from the group consisting of proteins, polypeptides and peptides and is covalently attached to the EV membrane by palmitoylation, and/or N-myristoylation and wherein the at least one endoprotease-cleavable linker is at least one furin-cleavable linker.

13. A nucleic acid molecule, or one or more nucleic acid sequences comprising an oligonucleotide encoding a biologically active compound, at least one endoprotease-cleavable linker and an attachment moiety for covalent attachment to an extracellular vesicle (EV) membrane.

14. The nucleic acid molecule or one or more nucleic acid sequences according to claim 13, wherein the biologically active compound is a protein, polypeptide or peptide, the at least one endoprotease-cleavable linker is at least one furin-cleavable linker and/or at least one calpain-cleavable linker and the attachment moiety for covalent attachment to the EV membrane is for palmitoylation, and/or N-myristoylation.

15. A pharmaceutical composition comprising the EV according to claim 1 and a pharmaceutically acceptable carrier.

16. A method for producing extracellular vesicles (EVs) 2 the method comprising:
introducing the nucleic acid molecule or one or more nucleic acid sequences of claim 13 into a mammalian host cell;
allowing the host cell to produce EVs; and
isolating the produced EVs.

17. (canceled)

18. A mammalian host cell provided with the nucleic acid molecule or one or more nucleic acid of claim 13, optionally further provided with a nucleic acid molecule comprising an oligonucleotide encoding Vesicular Stomatitis Virus G (VSV G) protein or a homolog thereof, syncytin-1, syncytin-2, herpes simplex virus glycoprotein B (HSV gB), rabies viral glycoprotein, influenza virus hemagglutinin (HA), human immunodeficiency virus envelope glycoprotein gp160 (gp160), Caenorhabditis elegans Eff-1 and/or Caenorhabditis elegans Aff-1.

19. The method according to claim 16, wherein the host cell is selected from the group consisting of HEK293, HER911, PER.C6, CHO, MSC-1, MCF-7, MDA-MB-231, Huh7, A549, HepG2, THP-1, PC3, B16-F10, C2C12, RAW264.7, A549, NIH-3T3, CT26.WT, EL4, HCT116, SCC-7, PANC-1, 4T1, MC38, U937, Jurkat, K562, Raji, Ramos, dendritic cells, platelets, macrophages, mesenchymal stem cells (MSCs), adipose-derived stem cells (ADSCs), bone marrow-derived mesenchymal stem cells (BM-MSCs), placenta-derived mesenchymal stem cells (PD-MSCs), and umbilical cord-derived mesenchymal stem cells (UC-MSCs), induced pluripotent stem cells (iPSCs), cardiac-derived progenitor cells (CPCs), cardiomyocytes, endothelial cells, fibroblasts, Schwann cells, astrocytes, oligodendrocytes, neurons, myoblasts, chondrocytes, tenocytes, hepatocytes, glioblastoma cells, and pancreatic islet cells.

20. The method according to claim 16, wherein the host cell is an endoprotease knock-out host cell, and/or wherein the method is performed in the presence of an inhibitor of the endoprotease.

21. (canceled)

22. A method of treating or preventing a disease in an individual in need thereof, the method comprising:
administering the EV of claim 1 to the individual.
